SEQUENCE LISTING (1 sequence)

SEQ ID NO: 1 - protein, 750 aa, Homo sapiens
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

(19) United States
(12) Patent Application Publication
(10) Pub. No.: US 2015/0147339 A1
(43) Pub. Date: May 28, 2015

(54) BIOMARKERS FOR PSMA TARGETED THERAPY FOR PROSTATE CANCER

(71) Applicant: PSMA DEVELOPMENT COMPANY, LLC, TARRYTOWN, NY (US)

(72) Inventors: WILLIAM C. OLSON, YORKTOWN HEIGHTS, NY (US); VINCENT DIPIPPO, MAHOPAC, NY (US)

(73) Assignee: PSMA DEVELOPMENT COMPANY, LLC, TARRYTOWN, NY (US)

(21) Appl. No.: 14/542,316

(22) Filed: Nov. 14, 2014

Related U.S. Application Data
(60) Provisional application No. 61/994,990, filed on May 18, 2014.
(60) Provisional application No. 61/994,785, filed on May 16, 2014.
(60) Provisional application No. 61/933,279, filed on Jan. 29, 2014.
(60) Provisional application No. 61/932,227, filed on Jan. 27, 2014.
(60) Provisional application No. 61/904,797, filed on Nov. 15, 2013.

(51) Int. Cl. G01N 33/574; G06F 19/00; G06F 19/10

(52) U.S. Cl. CPC G01N 33/57434; G06F 19/10; G06F 19/3431; G01N 2800/52
USPC 424/156.1; 435/7.23; 702/19

(57) ABSTRACT

Provided herein are a number of methods, assays and diagnostic tests based on one or more biomarkers as well as related kits and compositions that can be used to identify subjects or patients that would likely benefit from a treatment (or continued treatment), such as a PSMA targeted therapy. Also provided are methods for treating the identified subjects or patients.

### RELATED APPLICATIONS

This application claims the benefit under 35 U.S.C. §119 of U.S. provisional application 61/994,990, filed May 18, 2014, 61/994,785, filed May 16, 2014, 61/933,279, filed Jan. 29, 2014, 61/932,227, filed Jan. 27, 2014, and 61/904,797, filed Nov. 15, 2013, the entire contents of each of which are incorporated herein by reference.

### BACKGROUND OF THE INVENTION

Prostate cancer is the second leading cause of cancer death in American men. According to estimates from the American Cancer Society, it was expected for 2012 that approximately 241,740 new cases of prostate cancer would be diagnosed and 28,170 men would die of the disease in the United States (1). Today, because of increased screening and monitoring for prostate specific antigen (PSA), more than 90% of all prostate cancers are diagnosed in the local (i.e., confined to the prostate gland) or regional (i.e., confined to lymph nodes in the region of the prostate gland) stage. For patients with localized prostate cancer, primary treatment includes radical prostatectomy, external-beam radiation therapy, brachytherapy, or watchful waiting (1). Of these patients, 30% to 40% will fail local therapy (2).

Androgen-deprivation therapy (ADT), (e.g., hormone therapy), is the standard of care for subjects failing primary therapy (1). However, in nearly all patients, the tumor becomes castration resistant. There is no curative therapy for metastatic castration-resistant prostate cancer (mCRPC). Options for first-line therapy include abiraterone in combination with prednisone and docetaxel in combination with prednisone. Sipuleucel-T an autologous cellular immunotherapy, is indicated for the treatment of asymptomatic or minimally symptomatic mCRPC. Options for chemotherapy-experienced patients include enzalutamide, abiraterone and cabazitaxel in combination with prednisone (4-8). Radium 223 dichloride is indicated for the treatment of patients with castration-resistant prostate cancer, symptomatic bone metastases and no known visceral metastatic disease.

It has been found that prostate cancer is both heterogeneous and adaptable. Whereas most cases of prostate cancer originate as adenocarcinoma, a small percentage of tumors arise de novo from progenitor neuroendocrine cells within the prostate. Neuroendocrine cells produce specific proteins, such as neuron specific enolase (NSE), chromogranin A (CgA), bombesin, serotonin, somatostatin, a thyroid-stimulating-like peptide, parathyroid hormone-related peptides, and calcitonin which are secreted into the blood stream (9). Small-cell or neuroendocrine prostate cancer (NEPC) is an aggressive subtype that is associated with poor prognosis. Unlike adenocarcinoma, NEPC is unresponsive to androgen ablation and poorly susceptible to docetaxel-based chemotherapy. NEPC also expresses little-to-no PSA or PSMA. Neuroendocrine (NE) differentiation is one of the putative explanations for the development of castration-resistant disease. It is believed that NEPC emerges over time following transdifferentiation of adenocarcinoma tumors, particularly after prolonged periods of androgen suppression (4-8). Some researchers have speculated that the prevalence of NEPC may increase with the introduction of more potent antiandrogens (10).

### SUMMARY OF THE INVENTION

In one aspect, a companion diagnostic test comprising obtaining one or more biological samples from a subject undergoing a treatment or considered for a treatment; assaying the sample for a panel of biomarkers; generating a score with an algorithm based on the assay results of said panel of biomarkers; and determining the likely responsiveness of said subject to said treatment based on the results or score is provided. In some embodiments of any one or combination of the diagnostic tests, methods or assays provided herein, the diagnostic test further comprises isolating the biological sample prior to treatment. In some embodiments of any one or combination of the diagnostic tests, methods or assays provided herein, at least one of the biological sampled is obtained at baseline. In some embodiments of any one or combination of the diagnostic tests, methods or assays provided herein, at least one of the biological samples is obtained prior to treatment.

In some embodiments of any one or combination of the diagnostic tests, methods or assays provided herein, the panel of biomarkers comprises serum neuroendocrine markers. In some embodiments, the serum neuroendocrine markers are chromogranin A (CgA) and neuron-specific enolase (NSE).

In some embodiments of any one or combination of the diagnostic tests, methods or assays provided herein, the algorithm determines whether or not the sample exhibited low neuroendocrine levels. In some embodiments of any one or combination of the diagnostic tests, methods or assays provided herein, the algorithm determines whether or not the sample exhibited high neuroendocrine levels. As provided herein, low neuroendocrine levels can be indicative of a subject being likely responsive to a treatment. High neuroendocrine levels on the other hand can be indicative of a subject not being likely responsive to a treatment.

In some embodiments of any one or combination of the diagnostic tests, methods or assays provided herein, the algorithm comprises CgA patient assay value<3× Upper Limit of Normal (ULN), and in combination NSE patient assay value<1.5 ULN, equals low neuroendocrine levels. In some embodiments of any one or combination of the diagnostic tests, methods or assays provided herein, the algorithm comprises CgA patient assay value≦3×ULN, and in combination NSE patient assay value≦1.5 ULN, equals low neuorendocrine levels. In some embodiments of any one or combination of the diagnostic tests, methods or assays provided herein, a CgA patient value<3×5 nmole/L, and in combination a NSE patient value<1.5×12.5 ng/mL, equals low neuroendocrine levels.

In some embodiments of any one or combination of the diagnostic tests, methods or assays provided herein, the algorithm comprises CgA patient assay value>3×ULN, and in combination NSE patient assay value>1.5 ULN, equals high neuroendocrine levels. In some embodiments of any one or combination of the diagnostic tests, methods or assays provided herein, a CgA patient value>3×5 nmole/L, and in combination NSE patient value>1.5×12.5 ng/mL, equals high neuroendocrine levels.

In some embodiments of any one or combination of the methods or tests provided herein, the panel of biomarkers further comprises Prostate Serum Antigen (PSA). As provided herein, low neuroendocrine levels in combination with high PSA can be indicative of a subject being responsive to a treatment. In some embodiments of any one or combination of the diagnostic tests, methods or assays provided herein, a PSA value>than about 100 ng/mL equals high PSA. In some embodiments of any one or combination of the diagnostic tests, methods or assays provided herein, a PSA value>100 ng/mL equals high PSA. In some embodiments of any one or combination of the diagnostic tests, methods or assays provided herein, a PSA value<about 100 ng/mL can be indicative that a subject is not responsive or would not be responsive to a treatment. In some embodiments of any one or combination of the diagnostic tests, methods or assays provided herein, a PSA value<100 ng/mL can be indicative that a subject is not responsive or would not be responsive to a treatment.

In some embodiments of any one or combination of the diagnostic tests, methods or assays provided herein, the algorithm comprises CgA patient assay value<3×ULN, and in combination NSE patient assay value<1.5 ULN, equals low neuroendocrine levels; and patient PSA value>about 100 ng/mL. In some embodiments of any one or combination of the diagnostic tests, methods or assays provided herein, the algorithm comprises CgA patient assay value≦3×ULN, and in combination NSE patient assay value≦1.5 ULN, equals low neuroendocrine levels; and patient PSA value>about 100 ng/mL. In some embodiments of any one or combination of the diagnostic tests, methods or assays provided herein, the algorithm comprises CgA patient assay value≦3×ULN, and in combination NSE patient assay value≦1.5 ULN, equals low neuroendocrine levels; and wherein patient PSA value>100 ng/mL.

In some embodiments of any one or combination of the diagnostic tests, methods or assays provided herein, the panel of biomarkers further comprises PSMA intensity. In some embodiments of any one or combination of the diagnostic tests, methods or assays provided herein, the PSMA intensity is determined with an immunohistochemistry (IHC) procedure. In some embodiments of any one or combination of the diagnostic tests, methods or assays provided herein, the PSMA intensity is determined with an IHC procedure and determining an H-score, such as from a tumor tissue obtained from the subject at baseline or prior to treatment. As provided herein, a high H-score (i.e., H-score ≧than 200) correlated with the response to PSMA-ADC. In some embodiments of any one or combination of the diagnostic tests, methods or assays provided herein, the H-score value for comparison is 200. In some embodiments of any one or combination of the diagnostic tests, methods or assays provided herein, the H-score is equal to or greater than 200 and is indicative of responding to a treatment. In some embodiments of any one or combination of the diagnostic tests, methods or assays provided herein, the H-score is greater than 200 and is indicative of responding to a treatment. In some embodiments of any one or combination of the diagnostic tests, methods or assays provided herein, the H-score is less than 200 and is indicative of no or less of a response to a treatment. In some embodiments of any one or combination of the diagnostic tests, methods or assays provided herein, the H-score is calculated using the following formula:

H-score=(% cells showing 3+staining intensity)×3+(% cells showing 2+staining intensity)×2+(% cells showing 1+staining intensity).

In some embodiments of any one or combination of the diagnostic tests, methods or assays provided herein, the algorithm comprises CgA subject assay value≦3×ULN and NSE subject assay value≦1.5 ULN, equals low neuroendocrine levels; PSA value>100 ng/mL; and an H-score ≧than 200.

In some embodiments of any one or combination of the diagnostic tests, methods or assays provided herein, the panel of biomarkers further comprises Circulating Tumor Cells (CTCs). In some embodiments of any one or combination of the diagnostic tests, methods or assays provided herein, the CTCs are PSMA-expressing CTCs (PSMA+ CTCs). In some embodiments of any one or combination of the diagnostic tests, methods or assays provided herein, the PSMA+ CTCs are determined using an anti-PSMA antibody. In some embodiments of any one or combination of the diagnostic tests, methods or assays provided herein, the anti-PSMA antibody is PSMA 3.9 (ATCC PTA-3258).

In some embodiments of any one or combination of the diagnostic tests, methods or assays provided herein, the tests, methods or assays can also comprise performing a cell surface PSMA density assay. It has been found that patients (or subjects) that can likely benefit from treatment, such as a PSMA targeted therapy, can include those whose biological samples exhibited high density PSMA expression on CTC cells. In some embodiments of any one or combination of the methods or tests provided herein, the PSMA density for such a patient is >100,000 molecules of PSMA/PSMA⁺ CTC. In some embodiments of any one or combination of the methods or tests provided herein, the PSMA density for such a subject is >3+ average cell fluorescence intensity on a scale of zero to 4+ fluorescence intensity and the neuroendocrine level is low. The low neuroendocrine level may be defined as any one or combination of the levels provided herein described as a low neuroendocrine level.

In some embodiments of any one or combination of the diagnostic tests, methods or assays provided herein, the panel of biomarkers further comprises cell surface PSMA density. In some embodiments of any one or combination of the diagnostic tests, methods or assays provided herein, the algorithm comprises cell surface PSMA density>100,000 molecules of PSMA/PSMA+ CTC, equals high cell surface PSMA density. In some embodiments of any one or combination of the diagnostic tests, methods or assays provided herein, the algorithm comprises cell surface PSMA density>3+ average cell fluorescence intensity on a scale of zero to 4+ fluorescence intensity, equals high cell surface PSMA density, and the neuroendocrine level is low.

The PSMA density may be measured by any of a number of techniques known to those of ordinary skill in the art. For example, the density in some embodiments is measured by mean fluorescence intensity (MFI) using an automated flow analyzer. In some embodiments of any one or combination of the diagnostic tests, methods or assays provided herein, the cell surface PSMA density is measured by mean fluorescence intensity (MFI). In some embodiments of any one or combination of the diagnostic tests, methods or assays provided herein, the MFI=(average intensity of a foreground−average intensity of a background)×100. In some embodiments of any one or combination of the diagnostic tests, methods or assays provided herein, the algorithm comprises MFI>24, equals high cell surface PSMA density, and the neuroendocrine level is low. In some embodiments of any one or combination of the diagnostic tests, methods or assays provided herein, therefore, the MFI of PSMA⁺ CTCs in a subject that would likely benefit from a treatment, such as a PSMA targeted therapy, is >24 and the neuroendocrine level is low and the PSA is high. Such determinations may be made at baseline or prior to treatment. Again, a low neuroendocrine level may be as defined as any one or combination of the low neuroendocrine levels provided herein.

In some embodiments of any one or combination of the diagnostic tests, methods or assays provided herein, a score at baseline of low neuroendocrine levels, and high PSA, is indicative of likely responsiveness to treatment.

In some embodiments of any one or combination of the diagnostic tests, methods or assays provided herein, a score at baseline of low neuroendocrine levels, high PSA, and high PSMA intensity or high cell surface PSMA density on CTCs or tumor tissue, is indicative of likely responsiveness to treatment.

In some embodiments of any one or combination of the diagnostic tests, methods or assays provided herein, the tests, method or assays provide a predictive or likely response to a treatment, in particular, a predictive or likely response to a treatment comprising a PSMA-targeted therapy. Thus, the values obtained from any one or combination of diagnostic tests, methods or assays provided herein allow a physician to select an appropriate treatment for a subject. In some embodiments of any one or combination of the tests, methods or assays provided, a predicted or likely positive responsiveness to a treatment is a radiologic response, a decline in CTCs from baseline, a PSA decline from baseline, or a combination of two or more of the foregoing. In some embodiments of any one or combination of the diagnostic tests, methods or assays provided herein, the radiologic response is determined using RECIST criteria. In some embodiments of any one or combination of the diagnostic tests, methods or assays provided herein, a predicted or likely positive responsiveness is a radiologic response that is stable disease (SD), partial response (PR) or complete response (CR).

In some embodiments of any one or combination of the diagnostic tests, methods or assays provided herein, the subject has prostate cancer. In some embodiments of any one or combination of the diagnostic tests, methods or assays provided herein, the subject has metastatic castration-resistant prostate cancer (mCRPC). In some embodiments of any one or combination of the diagnostic tests, methods or assays provided herein, the subject was previously treated with at least one taxane and progressed despite treatment. In some embodiments of any one or combination of the diagnostic tests, methods or assays provided herein, the subject was previously treated with abiraterone and/or enzalutamide and progressed despite treatment. In some embodiments of any one or combination of the diagnostic tests, methods or assays provided herein, the subject was previously treated with at least one taxane and at least one anti-androgen and progressed despite treatment. In some embodiments of any one or combination of the diagnostic tests, methods or assays provided herein, the subject has not received prior chemotherapy.

In some embodiments of any one or combination of the diagnostic tests, methods or assays provided herein, the treatment is a PSMA targeting treatment that comprises a PSMA ligand-anticancer agent conjugate. In some embodiments of any one or combination of the diagnostic tests, methods or assays provided herein, the treatment comprises a PSMA antibody-drug conjugate (PSMA ADC). In some embodiments of any one or combination of the diagnostic tests, methods or assays provided herein, the drug is an inhibitor of tubulin polymerization. In some embodiments of any one or combination of the diagnostic tests, methods or assays provided herein, the drug is an auristatin derivative. In some embodiments of any one or combination of the diagnostic tests, methods or assays provided herein, the auristatin derivative is monomethylauristatin norephedrine (MMAE) or monomethylauristatin phenylalanine (MMAF).

In some embodiments of any one or combination of the diagnostic tests, methods or assays provided herein, the PSMA ADC is administered at 1.8 mg/kg, 2.0 mg/kg, 2.2 mg/kg, 2.3 mg/kg, 2.4 mg/kg, or 2.5 mg/kg intravenously. In some embodiments of any one or combination of the diagnostic tests, methods or assays provided herein, the PSMA ligand of the conjugate comprises a small molecule ligand that binds specifically PSMA. In some embodiments of any one or combination of the diagnostic tests, methods or assays provided herein, the small molecule ligand binds an enzymatic site on PSMA.

In some embodiments of any one or combination of the diagnostic tests, methods or assays provided herein, the test, method or assay is used to select a subject (or patient) likely to benefit from the treatment, such as a PSMA targeted treatment. In some embodiments of any one or combination of the diagnostic tests, methods or assays provided herein, the treatment is administered or information regarding the treatment is provided to the subject when the subject is determined to be or likely to be responsive to the treatment. In some embodiments of any one or combination of the diagnostic tests, methods or assays provided herein, the selected patient for PSMA targeted treatment has low neuroendocrine levels (as defined anywhere herein in some embodiments) and a PSA level of >100 ng/mL.

In some embodiments of any one or combination of the diagnostic tests, methods or assays provided herein, the selected patient is administered a PSMA ligand-anticancer agent conjugate. In some embodiments of any one or combination of the diagnostic tests, methods or assays provided herein, the PSMA ligand-anticancer agent conjugate is a PSMA ADC. In some embodiments of any one or combination of the diagnostic tests, methods or assays provided herein, the drug is an inhibitor of tubulin polymerization. In some embodiments of any one or combination of the diagnostic tests, methods or assays provided herein, the drug is an auristatin derivative. In some embodiments of any one or combination of the diagnostic tests, methods or assays provided herein, the auristatin derivative is monomethylauristatin norephedrine (MMAE) or monomethylauristatin phenylalanine (MMAF). In some embodiments of any one or combination of the diagnostic tests, methods or assays provided herein, the PSMA ADC is administered at 1.8 mg/kg, 2.0 mg/kg, 2.2 mg/kg, 2.3 mg/kg, 2.4 mg/kg, or 2.5 mg/kg intravenously. In some embodiments of any one or combination of the diagnostic tests, methods or assays provided herein, the PSMA ligand of the conjugate comprises a small molecule ligand that binds specifically PSMA. In some embodiments of any one of the diagnostic tests, methods or assays provided herein, the small molecule ligand binds an enzymatic site on PSMA.

In some embodiments of any one or combination of the diagnostic tests, methods or assays provided herein, based on the results of the methods or diagnostic tests provided herein, a treatment other than a PSMA targeted treatment may be indicated. In some embodiments of any one or combination of the diagnostic tests, methods or assays provided herein, the treatment comprises at least one Aurora Kinase inhibitor (e.g., an inhibitor of an Aurora kinase, which regulates cell cycle transit from G2 through cytokinesis). In some embodiments of any one or combination of the diagnostic tests, methods or assays provided herein, the Aurora Kinase inhibitor is PHA-739358 (Danusertib), CYC116, SNS-314, AT9283, R763, PF-03814735, GSK1070916, AMG-900, AZD-1152, or Hesperidin.

In some embodiments of any one or combination of the diagnostic tests, methods or assays provided herein, the selected patient has high neuroendocrine levels (as defined anywhere herein in some embodiments) and a PSA level of <100 ng/mL, and said selected patient is administered at least one Aurora Kinase inhibitor. In some embodiments of any one or combination of the diagnostic tests, methods or assays provided herein, the Aurora Kinase inhibitor is selected from the group consisting of PHA-739358 (Danusertib), CYC116, SNS-314, AT9283, R763, PF-03814735, GSK1070916, AMG-900, AZD-1152, and Hesperidin. Other Aurora Kinase inhibitors are known in the art (Invest New Drugs (2012) 30:2411-2432).

In some aspects, methods, assays or tests are provided for identifying a subject that will likely benefit from a treatment, such as a PSMA targeted therapy, as provided herein. The methods, assays or tests can include any one or more (including all) of the steps as provided in any one of the methods, assays or tests described. In some embodiments of any one or combination of the methods, assays or tests provided, the methods or assays comprise determining the average cell surface PSMA density on PSMA⁺CTCs.

In some aspects, a method of treating a PSMA expressing cancer is provided. In some embodiments of any one or combination of the diagnostic tests, methods or assays provided herein, the PSMA expressing cancer is prostate cancer. In some embodiments of any one or combination of the diagnostic tests, methods or assays provided herein, the prostate cancer is metastatic prostate cancer. In some embodiments of any one or combination of the diagnostic tests, methods or assays provided herein, the prostate cancer is castration-resistant metastatic prostate cancer. In some embodiments of any one or combination of the diagnostic tests, methods or assays provided herein, the subject may be any one of the subjects described herein.

In some embodiments of any one or combination of the methods provided herein, the method can comprise performing a biomarker test on a patient sample before treatment or before continued treatment (at baseline); and providing a treatment likely to benefit the patient according to the results of the biomarker test. The biomarker test can be any one of the tests or assays provided herein and can, therefore, be any one of the companion diagnostic tests provided herein.

In some aspects, methods of treating prostate cancer in a patient (or subject) is provided. In some embodiments of any one or combination of the methods provided, the prostate cancer can be any one of the types of prostate cancer provided herein. In some embodiments of any one or combination of the methods provided, the method comprises testing for any one or more of the biomarkers provided herein in a biological sample from the patient and administering a therapeutically effective amount of any one of the treatments provided herein to the patient if the sample meets any or more of the criteria provided herein for the one or more biomarkers. In some embodiments of any one or combination of the methods provided, the testing can be performed according to any one or combination of the methods, assays or tests provided herein.

In some aspects, methods of identifying patients (or subjects) with prostate cancer eligible for treatment with any one of the treatments provided herein is provided. In some embodiments of any one or combination of the methods provided, the prostate cancer can be any one of the types of prostate cancer provided herein. In some embodiments of any one or combination of the methods provided, the method comprises testing a biological sample from the patient for one or more of the biomarkers provided herein, wherein the patient is eligible for the treatment if the sample meets any one or more of the criteria provided herein for the one or more biomarkers. In some embodiments of any one or combination of the methods provided, the testing can be performed according to any one of the methods or tests provided herein.

In some embodiments of any one or combination of the diagnostic tests, methods or assays provided herein, the biomarker test is a test that assays one or more neuroendocrine enzymes. In some embodiments of any one or combination of the diagnostic tests, methods or assays provided herein, the neuroendocrine enzymes comprise serum Chromogranin A (CgA) and/or serum neuron-specific enolase (NSE). In some embodiments of any one or combination of the diagnostic tests, methods or assays provided herein, the test further comprises an assay serum PSA. In some embodiments of any one or combination of the diagnostic tests, methods or assays provided herein, the biomarker test further comprises a PSMA intensity assay. In some embodiments of any one or combination of the diagnostic tests, methods or assays provided herein, the PSMA intensity assay is an IHC procedure and determining an H-score. In some embodiments of any one or combination of the diagnostic tests, methods or assays provided herein, the H-score is calculated according to the following formula:

H-score=(% cells showing 3+staining intensity)×3+(% cells showing 2+staining intensity)×2+(% cells showing 1+staining intensity).

In some embodiments of any one or combination of the diagnostic tests, methods or assays provided herein, an H-score ≧200, equals a high PSMA intensity.

In some embodiments of any one or combination of the diagnostic tests, methods or assays provided herein, the biomarker test further assays CTCs. In some embodiments of any one or combination of the diagnostic tests, methods or assays provided herein, the biomarker test can further assay PSMA expressing CTCs (PSMA⁺ CTCs). In some embodiments of any one or combination of the methods or tests provided herein, the biomarker test can further assay cell surface PSMA density, such as on CTCs.

As provided elsewhere herein, the density can be measured by any of a number of methods known to those of ordinary skill in the art. In some embodiments of any one or combination of the diagnostic tests, methods or assays provided herein, the density is measured by mean fluorescence intensity (MFI).

In some embodiments of any one or combination of the diagnostic tests, methods or assays provided herein, the results of the test are indicative of a low neuroendocrine value (as defined anywhere herein in some embodiments) and the subject is likely to benefit from treatment, such as with a PSMA targeted therapy. In some embodiments of any one or combination of the diagnostic tests, methods or assays provided herein, a low endocrine value is a CgA patient assay value<3×ULN, in combination with a NSE patient assay value<1.5 ULN. In some embodiments of any one or combination of the diagnostic tests, methods or assays provided herein, a low endocrine value is a CgA patient assay value≦3×ULN, in combination with a NSE patient assay value≦1.5 ULN.

In some embodiments of any one or combination of the diagnostic tests, methods or assays provided herein, the results of the test are indicative of a high PSA value (as defined anywhere herein in some embodiments) and can also be indicative that the subject is likely to benefit from treatment, such as with a PSMA targeted therapy. In some embodiments of any one or combination of the diagnostic tests, methods or assays provided herein, a high PSA value is a PSA value that >about 100 ng/mL. In some embodiments of any one or combination of the diagnostic tests, methods or assays provided herein, a high PSA value is a PSA value that >100 ng/mL In some embodiments of any one or combination of the diagnostic tests, methods or assays provided herein, a high PSA value in combination with a low neuroendocrine value is indicative that a subject is likely to benefit from treatment, such as with a PSMA targeted therapy. In some embodiments of any one or combination of the diagnostic tests, methods or assays provided herein, a high PSA value in combination with a low neuroendocrine value; and a high PSMA intensity or high cell surface PSMA density on CTCs or tumor tissue, is indicative that a subject is likely to benefit from treatment, such as with a PSMA targeted therapy. The high PSA value and low neuroendocrine; and high PSMA intensity or cell surface PSMA density on CTCs or tumor tissue, respectively, can each be any one of the levels provided herein. The therapy can be any one of the therapies or treatment provided herein.

In some embodiments of any one or combination of the diagnostic tests, methods or assays provided herein, when the results are a high neuroendocrine value and a PSA value<100 ng/ml, no treatment is given or recommended, an alternative treatment is given or recommended or the treatment given or recommended is watchful waiting.

In some embodiments of any one or combination of the diagnostic tests, methods or assays provided herein, the results of the test indicate a high neuroendocrine value (as anywhere defined herein in some embodiments) and a PSA value<100 ng/ml. In some embodiments of any one or combination of the diagnostic tests, methods or assays provided herein, these results can indicate that the subject would be likely to benefit from the administration of a treatment that is not a PSMA targeted therapy such as with the administration of at least one Aurora Kinase inhibitor. The Aurora Kinase inhibitor can be any one of such inhibitors described herein.

The subjects that can be treated or assessed by any one of the tests, methods or assays provided herein, may be any of the subjects described herein. Such subjects include those that have prostate cancer, such as progressive metastatic castration-resistant prostate cancer. The subjects that can be treated or assessed by any one of the tests, methods or assays provided herein, may be a subject that has had prior chemotherapy with at least one taxane. In some embodiments of any one or combination of the assays, methods or tests provided herein, the taxane is selected from the group consisting of docetaxel, cabazitaxel, and combinations thereof. In some embodiments of any one or combination of the assays, methods or tests provided herein, the subject may also be one that has had prior treatment with one or more antiandrogens. In some embodiments of any one or combination of the assays, methods or tests provided herein, the antiandrogens are enzalutamide, abiraterone, or combinations thereof. In some embodiments of any one or combination of the assays, methods or tests provided herein, the subject was previously treated with at least one taxane and at least one antiandrogen. In some embodiments of any one or combination of the assays, methods or tests provided herein, the cancer has progressed despite prior treatment in the subjects that can be treated or assessed by any one of the assays, methods or tests provided herein.

In some embodiments of any one or combination of the methods, tests or assays provided, the method, test or assay comprises obtaining a biological sample from a subject undergoing a treatment or is considered for a treatment; conducting a PSMA expressing CTC assay; determining an average cell surface PSMA density; determining the likely responsiveness of the subject to the treatment based on the results. In some embodiments of any one or combination of the methods, tests or assays provided, the biological sample is isolated prior to treatment. In some embodiments of any one or combination of the methods, tests or assays provided, the determining is based on an average density score. In some embodiments of any one or combination of the methods, tests or assays provided, the score is >100,000 molecules of PSMA per PSMA⁺ CTC. In some embodiments of any one of the methods, tests or assays provided, the PSMA density is >3+ average cells fluorescence intensity on a scale of zero to 4+ fluorescence intensity.

The density can be determined by any of the methods known to those of ordinary skill in the art. Any of such methods can be used to determine if the subject is predicted to benefit from a treatment. In some embodiments of any one or combination of the methods, assays or tests provided, the density is measured by mean fluorescence intensity (MFI) of a fluorescein labeled-PSMA binding reagent. In some embodiments of any one or combination of the methods, assays or tests provided, the density score is a MFI>24. In some embodiments of any one of the methods, assays or tests provided, when the MFI is >24, the subject is selected for treatment with a PSMA targeted therapy, such as with a PSMA ligand-anticancer agent conjugate. In some embodiments of any one or combination of the methods, assays or tests provided, the density score is a MFI<24. In some of these embodiments, the subject is not selected for treatment with a PSMA targeted therapy.

In another aspect biomarker assays are provided. Such assays may be for identifying whether or not a subject will likely to respond to a PSMA targeted therapy. In some embodiments, that assay comprises determining the average cell surface PSMA density on PSMA+ CTCs. In some embodiments of any one or combination of the assays, methods or tests provided herein the PSMA targeted therapy can comprise any one of the PSMA targeted treatments provided herein.

In some embodiments of the assay, the assay comprises the steps obtaining a biological sample from a subject undergoing a treatment or being considered for a treatment; conducting a PSMA expressing CTC assay; determining an average cell surface PSMA density; and determining the likely responsiveness of the subject to the treatment based on the results. In some embodiments of any one or combination of the assays provided, the biological sample is obtained prior to treatment.

In some embodiments of any one or combination of the assays provided, the likely responsiveness is based on the average cell surface PSMA density score. In some embodiments of any one or combination of the assays provided, an average cell surface PSMA density score>100,000 molecules of PSMA/PSMA+ CTCs is a high average score. In some embodiments of any one or combination of the assays provided, an average cell surface PSMA density score is >3+ average cell fluorescence intensity on a scale of zero to 4+ fluorescence intensity is a high average score. In some embodiments of any one or combination of the assays provided, the average cell surface PSMA density is measured by MFI. In some embodiments of any one or combination of the assays provided, the average cell surface PSMA density is measured by MFI of a fluorescein labeled-PSMA binding agent. In some embodiments of any one or combination of the assays provided, the PSMA binding agent is PSMA 3.9 (ATCC PTA-3258). In some embodiments of any one or combination of the assays provided, MFI=(average intensity of a foreground−average intensity of a background)×100. In some embodiments of any one or combination of the assays provided, when the density score is a MFI>24 there is a high average cell surface PSMA density. In some embodiments of any one or combination of the assays provided, when density score is a MFI<24 there is not a high average cell surface PSMA density.

In some embodiments of any one or combination of the assays provided, the subject is selected for treatment or continued treatment as provided herein. In some embodiments of any one or combination of the assays provided, the subject is predicted to benefit from a treatment. In some embodiments of any one or combination of the assays provided, the subject is not selected for treatment with a PSMA ligand-anticancer agent conjugate.

In some embodiments of any one or combination of the assays provided, the assay is for determining whether or not a subject will likely to respond to a PSMA targeted therapy and the assay comprises determining PSMA expression in a sample from a subject using an IHC procedure. In some embodiments of any one or combination of the assays provided, the assay comprises determining an H-score. In some embodiments of any one of the assays provided, the H-score is calculated according to the following formula:

H-score=(% cells showing 3+staining intensity)×3+(% cells showing 2+staining intensity)×2+(% cells showing 1+staining intensity).

In some embodiments of any one or combination of the assays provided, when the H-score is ≧than 200, the H-score is a high H-score. In some embodiments of any one or combination of the assays provided, the subject is deemed responsive to a PSMA targeted therapy. In some embodiments of any one or combination of the assays provided, the subject is selected for treatment or continued treatment as provided herein. In some embodiments of any one or combination of the assays provided, the subject is predicted to benefit from a treatment as provided herein. In some embodiments of any one or combination of the assays provided, the subject is not selected for treatment or continued treatment with a PSMA ligand-anticancer agent conjugate. In some embodiments of any one or combination of the assays provided, when the H-score is less than 200 the subject is not selected for treatment or continued treatment with the PSMA ligand-anticancer agent conjugate or is selected for treatment or continued treatment with a therapeutic agent other than the PSMA ligand-anticancer agent conjugate.

In some embodiments of any one or combination of the assays provided, the assay comprises measuring one or more serum neuroendocrine markers in a sample from a subject. In some embodiments of any one or combination of the assays provided, the assay comprises determining a CgA subject assay value and a NSE subject assay value. In some embodiments of any one or combination of the assays provided CgA subject assay value≦3×(Upper Limit of Normal (ULN) and NSE subject assay value≦1.5 ULN, equals low neuroendocrine levels. In some embodiments of any one or combination of the assays provided, when the neuroendocrine level is low, the subject is deemed responsive to a PSMA targeted therapy. In some embodiments of any one or combination of the assays provided, the subject is selected for treatment or continued treatment as provided herein. In some embodiments of any one or combination of the assays provided, the subject is predicted to benefit from a treatment. In some embodiments of any one or combination of the assays provided, the subject is not selected for treatment or continued treatment with a PSMA ligand-anticancer agent conjugate.

In other aspects, diagnostic kits are provided that include one or more assay reagents that can be used to carry out any one of the assays, methods or tests provided herein or one or more steps thereof. In some embodiments of any one of the kits provided, the kit comprises the assay reagents of any one or combination of the tests, methods or assays provided herein or that would be used to carry one any one or combination of the tests, methods or assays provided herein. In some embodiments of any one of the kits provided, the kit is a diagnostic kit for selecting a prostate cancer patient for treatment, wherein the diagnostic kit comprises assay reagents to measure serum levels of neuroendocrine enzymes. In some embodiments of any one of the kits provided, the kit can further comprise instructions for selecting. In some embodiments of any one of the kits provided, a low neuroendocrine level is indicative that the patient is likely to benefit from treatment such as with a PSMA ligand-anticancer agent conjugate. The low neuroendocrine level may be any one of the levels provided herein. The kits provided herein can be used to assess the likelihood a patient (or subject) will benefit from any one of the treatments provided herein.

In some embodiments of any one of the kits provided, the assay is an immunoassay. In some embodiments of any one of the kits provided, the kit comprises reagents to conduct a serum Chromogranin A (CgA) assay and reagents to conduct a serum neuron-specific enolase (NSE) assay.

In some embodiments of any one of the kits provided, the kit further comprises reagents to measure serum levels of PSA, reagents to assess CTC, reagents to measure PSMA intensity, and/or reagents to determined PSMA density, such as on CTCs, or any combination thereof including all of the foregoing. In some embodiments of any one of the kits provided, the reagent that measures PSMA intensity or density is an anti-PSMA antibody, such as PSMA 3.9 (ATCC PTA-3258). In some embodiments of any one of the kits provided, the kit comprises or further comprises reagents for performing an IHC procedure and determining an H-score. In some embodiments of any one of the kits provided, the H-score is calculated according to the following formula:

H-score=(% cells showing 3+staining intensity)×3+(% cells showing 2+staining intensity)×2+(% cells showing 1+staining intensity).

In some embodiments of any one of the kits provided, a kit that includes a reagent for determining PSMA density or intensity such as on PSMA⁺ CTCs or tumor tissue includes a fluorochrome.

In some aspects, a kit, such as a biomarker kit, for selecting a prostate cancer patient predicted to benefit from a treatment, the kit comprising reagents for use in an biomarker assay to determine the PSMA density on PSMA⁺ CTCs in a biological sample obtained from the patient is provided. In some embodiments of any one of the kits provided, the treatment comprises a PSMA targeted therapy such as a PSMA ligand-anticancer conjugate. In some embodiments of any one of the kits provided herein the kit can further comprise instruction for selecting. In some embodiments of any one of the kits provided, the assay is an immunoassay. In some embodiments of any one of the kits provided, the assay uses a fluorochrome and the kit may comprise a fluorochrome such as phycoerythrin.

In some embodiments of any one or combination of the tests, methods, assays or kits provided herein, when there is an assay for measuring CTCs, the assay for measuring CTCs comprises (1) placing nucleated cells from blood samples onto slides, (2) storing the slides, optionally in a −80° C. biorepository, (3) staining the slides with specific binding reagents to identify one or more CTC markers and PSMA, said reagents having a detectable label, (4) scanning the slides for one or more detectable labels, (5) running one or more multi-parametric digital pathology algorithms and (6) detection of CTCs and quantitation of biomarker expression.

In some embodiments of any one or combination of the tests, methods, assays or kits provided herein, when there is an assay for measuring CTCs, the assay for measuring CTCs comprises (1) obtaining one or more whole blood samples, (2) staining with one or more specific binding reagents to identify one or more CTC markers and PSMA, said reagents having a detectable label, (3) scanning for one or more detectable labels, (5) running one or more algorithms and (6) detection of CTCs and quantitation of biomarker expression.

In some embodiments of any one or combination of the tests, methods, assays or kits provided herein, the assay for measuring CTCs is any one of the assays described herein including those in the Examples and/or in the Figures.

In some aspects, a test, method, assay or kit is provided, wherein the test, method, assay or kit is any one or combination of those described herein including those of the Examples and/or in the Figures.

In some embodiments of any one or combination of the tests, methods, assays or kits provided herein, the subject is a patient. In some embodiments of any one or combination of the tests, methods, assays or kits provided herein, the patient is a subject.

In some embodiments of any one or combination of the tests, methods, assays or kits provided herein, the treatment can be continuing treatment with the same type of therapy such as a PSMA targeted therapy.

In some embodiments of any one or combination of the tests, methods, assays or kits provided herein, the biological sample is a sample comprising PSMA⁺ cells, such as PSMA⁺ CTCs, or a tumor tissue sample.

### BRIEF DESCRIPTION OF THE DRAWINGS

FIG. 1 shows graphs of neuroendocrine (NE) biomarker metrics for patients treated with a 2.5 mg/kg dose of a prostate specific membrane antigen (PSMA) antibody-drug conjugate (ADC).

FIG. 2 shows a graph of circulating PSMA⁺ tumor cell (CTC) intensity and neuroendocrine (NE) correlations with prostate specific antigen (PSA) response obtained from evaluable patients who received greater than two 2.5 mg/kg doses of PSMA-ADC.

FIG. 3 shows a graph of PSMA⁺ CTC intensity and NE correlations with CTC response obtained from evaluable patients who received greater than two 2.5 mg/kg doses of PSMA-ADC and greater than or equal to five CTCs at baseline.

FIG. 4 shows graphs of PSA responses correlate with low neuroendocrine (NE), obtained from evaluable patients who received greater than two 2.5 mg/kg doses of PSMA ADC.

FIG. 5 shows graphs of CTC responses obtained from evaluable patients who received greater than two 2.5 mg/kg doses of PSMA ADC and who had greater than or equal to five CTCs at baseline correlated with low NE.

FIG. 6 shows a graph of PSMA⁺ CTC intensity and NE correlations with PSA responses obtained from evaluable patients who received greater than two 2.3* mg/kg doses of PSMA-ADC or greater than two 2.5 mg/kg doses of PSMA-ADC. *Interim analysis.

FIG. 7 shows a graph of PSMA⁺ CTC intensity and NE correlations with CTC response obtained from evaluable patients who received greater than two 2.3* mg/kg doses of PSMA-ADC and greater than or equal to five CTCs at baseline, or greater than two 2.5 mg/kg doses of PSMA-ADC and greater than or equal to five CTCs at baseline. *Interim analysis.

FIG. 8 shows graphs of PSA responses correlated with low NE obtained from evaluable patients who received greater than two 2.3* mg/kg doses of PSMA-ADC or greater than two 2.5 mg/kg doses of PSMA-ADC. *Interim analysis.

FIG. 9 shows graphs of CTC response obtained from evaluable patients who received greater than two 2.3* mg/kg doses of PSMA-ADC and who had greater than or equal to five CTCs at baseline, or greater than two 2.5 mg/kg doses of PSMA-ADC and who had greater than or equal to five CTCs at baseline. *Interim analysis.

FIG. 10 shows a graph of overall survival of treated patients. *Interim analysis.

FIG. 11 shows a digimizer analysis workflow diagram (Method 1).

FIG. 12 shows schematics of the CTC collection and detection process from Epic Sciences (CTC Method 2).

FIGS. 13A and 13B show graphs of antibody titration curves of rabbit monoclonal anti-PSMA antibody (CTC Method 2).

FIG. 14 shows microscopy images of PSMA staining in PC3 (no PSMA) and LNCaP (high PSMA) cells (CTC Method 2).

FIG. 15 shows a graph of specificity data obtained from an anti-PSMA antibody interaction assay (CTC Method 2).

FIG. 16 shows a graph of PSMA signal or intensity detection in banked CTC samples obtained from prior treated mCRPC patients (CTC Method 2).

FIG. 17 shows microscopy images of PSMA⁺ CTCs stained with the PSMA CTC assay Method 2.

FIG. 18 shows the Phase 2 study schematic.

FIG. 19 shows graphs of PSA and CTC results for samples obtained from all patients from the Phase 2 study.

FIG. 20 shows graphs of PSA and CTC results for samples obtained from patients with greater than or equal to median PSMA expression.

FIG. 21 shows graphs of PSA and CTC results for evaluable patients with low NE markers*. * CgA<3×ULN; NSE<1.5×ULN; PSA>100 ng/mL patients with baseline.

FIG. 22 shows a summary of PSMA ADC baseline characteristics. *Interim analysis.

FIG. 23 shows graphs of PSA and CTC results for patients with a high immunohistochemical (IHC) PSMA marker.

FIG. 24 shows a graph of best Response Evaluation Criteria In Solid Tumors (RECIST) target lesion change from baseline following treatment of chemo-experienced patients with PSMA ADC (end of study).

FIG. 25 shows efficacy of PSMA ADC treatment in chemo-experienced and chemo-naïve patients as demonstrated by radiological response in patients with measurable target lesions correlated with PSA and CTC responses.

FIG. 26A shows graphs of neuroendocrine biomarker metrics for patients who received the 2.3 mg/kg dose of PSMA-ADC and for patients who received the 2.5 mg/kg dose of PSMA-ADC. FIG. 26B shows graphs of neuroendocrine biomarker metrics for all patients, including chemotherapy-experienced and chemo-naïve patients.

FIG. 27A shows graphs of PSA responses in evaluable patients who received the 2.3 mg/kg dose of PSMA ADC and patients who received the 2.5 mg/kg dose of PSMA ADC (all evaluable chemo-experienced patients, left; evaluable chemo-experienced patients with low NE markers, right). FIG. 27B shows graphs of PSA responses in all evaluable chemo-experienced and chemo-naïve patients patients who received PSMA ADC (all evaluable, left; evaluable patients with low NE markers, right).

FIG. 28A shows graphs of CTC responses in evaluable patients who received the 2.3 mg/kg dose of PSMA ADC and patients who received the 2.5 mg/kg dose of PSMA ADC (all evaluable chemo-experienced patients, left; evaluable chemo-experienced patients with low NE markers, right). FIG. 28B shows graphs of CTC responses in all evaluable chemo-experienced and chemo-naïve patients patients who received PSMA ADC (all evaluable, left; evaluable patients with low NE markers, right).

FIG. 29A shows graphs of PSMA biomarker metrics for chemo-experienced patients who received the 2.3 mg/kg dose and for chemo-experienced patients who received the 2.5 mg/kg dose. FIG. 29B shows graphs of PSMA biomarker metrics for all evaluable chemo-experienced and chemo-naïve patients patients (≧5 CTCs at baseline).

FIG. 30A shows graphs of a PSMA biomarker analysis of PSA responses in evaluable patients who received 2.3 mg/kg doses of PSMA ADC and chemo-experienced patients who received 2.5 mg/kg doses of PSMA ADC (all evaluable patients, left; evaluable patients with high PSMA markers, right). FIG. 30B shows graphs of a PSMA biomarker analysis of PSA responses in all evaluable chemo-experienced and chemo-naïve patients who received PSMA ADC (all evaluable patients, left; evaluable patients with high PSMA markers, right).

FIG. 31A shows graphs of a PSMA biomarker analysis of CTC responses in evaluable chemo-experienced patients who received the 2.3 mg/kg dose of PSMA ADC and chemo-experienced patients who received the 2.5 mg/kg dose of PSMA ADC, ≧5 CTCs at baseline (all evaluable patients, left; evaluable patients with high PSMA markers, right). FIG. 31B shows graphs of a PSMA biomarker analysis of CTC responses in all evaluable chemo-experienced and chemo-naïve patients patients who received PSMA ADC, who had ≧5 CTCs at baseline (all evaluable patients, left; evaluable patients with high PSMA markers, right).

FIG. 32A shows a graph of PSMA intensity and NE correlations with PSA response in evaluable chemo-experienced patients who received the 2.3 mg/kg dose and chemo-experienced patients who received the 2.5 mg/kg dose. FIG. 32B shows a graph of PSMA intensity and NE correlations with PSA response in all evaluable chemo-experienced and chemo-naïve patients patients.

FIG. 33A shows a graph of PSMA intensity and NE correlations with CTC response in evaluable chemo-experienced patients who received the 2.3 mg/kg dose and chemo-experienced patients who received the 2.5 mg/kg dose. FIG. 33B shows a graph of PSMA intensity and NE correlations with CTC response in all evaluable chemo-experienced and chemo-naïve patients patients.

FIG. 34A shows a graph of overall survival of all patients treated with PSMA ADC (n=119), including chemotherapy-experienced and chemotherapy-naïve patients. FIG. 34B shows a graph of overall survival of all chemotherapy-experienced patients (n=83). FIG. 34C shows a percent comparison of all patients (n=119) with prior treatments and chemotherapy-experienced patients (n=83) with prior treatments, who went on to receive PSMA ADC treatment.

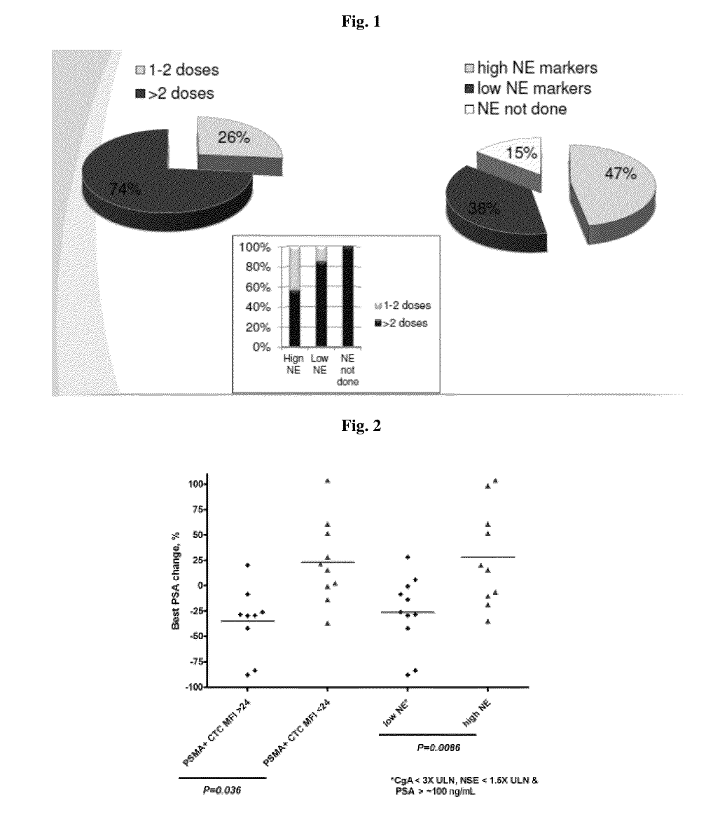

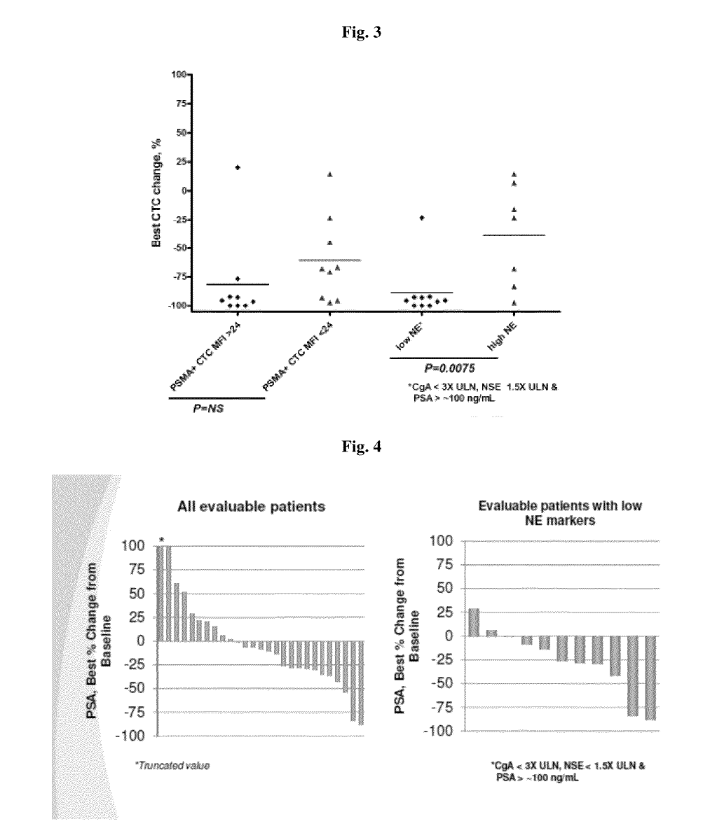

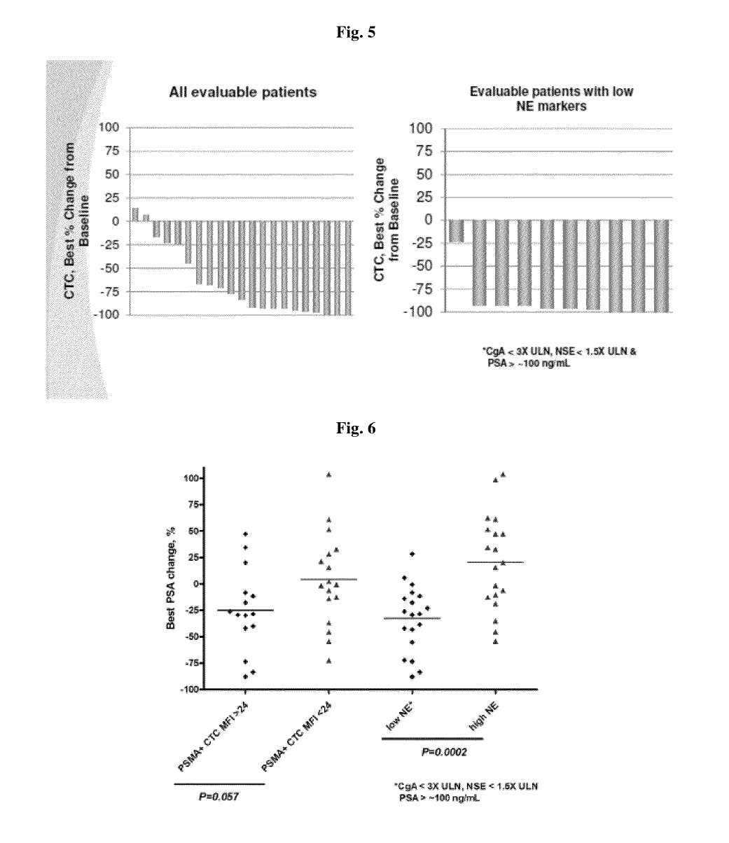

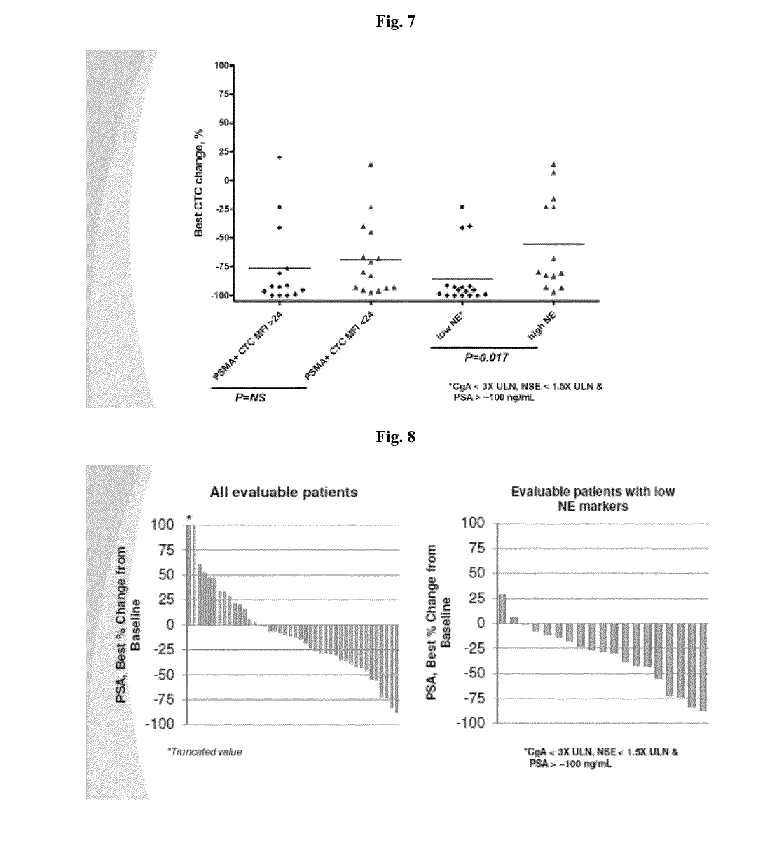

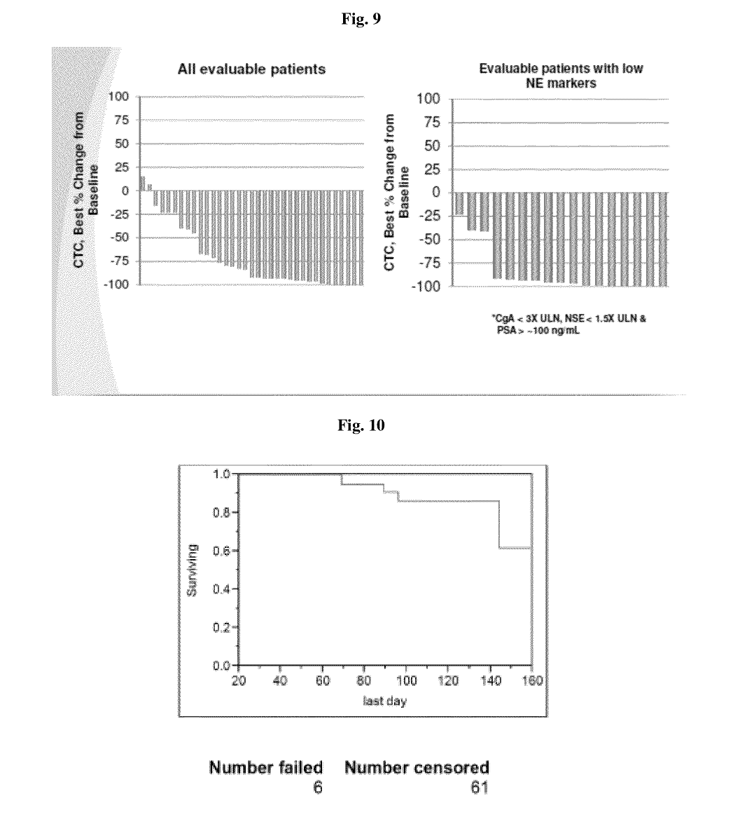

### DETAILED DESCRIPTION OF THE INVENTION

New therapies will expand therapeutic options for subjects with prostate cancer, such as mCRPC, to improve therapeutic outcome. One approach addressing this involves the use of monoclonal antibodies (mAb) to deliver cytotoxic agents to prostate tumor cells. PSMA ADC (prostate specific membrane antigen antibody-drug conjugate) therapy can target PSMA expressing cancer cells. Additionally, the identification of biomarkers in prostate cancer which are predictive of efficacy in a particular treatment modality in a patient, at a particular stage of disease will also be helpful. Such a biomarker would be useful as a companion diagnostic in the identification and selection of patients likely to benefit from a particular treatment. In particular, a biomarker in patients with advanced metastatic prostate cancer, after progression of disease despite treatment with taxanes and androgen deprivation, would be beneficial.

Taxanes are diterpenes produced by the plants of the genus Taxus (yews) as well as synthetic derivatives, and are widely used as chemotherapy agents. Taxane agents include paclitaxel (Taxol), docetaxel (Taxotere) and cabazitaxel (Jevtana®).

An “antiandrogen,” as used herein, refers to an agent that blocks (e.g., inhibits) the action of androgen hormones and androgen-regulated molecules. Androgen receptor antagonists are herein considered to be antiandrogens. The term “antiandrogen” includes antiandrogens, antiandrogen analogs, and antiandrogen derivatives. In prostate cancer, antiandrogens block the activity of testosterone, which typically slows prostate cancer growth. In some embodiments, an antiandrogen blocks enzyme cytochrome P450 17A1, encoded by the CYP17A gene. Antiandrogens may be steroidal or non-steroidal (also referred to as “pure”). Examples of antiandrogens for use as provided herein include, without limitation, abiraterone (ZYTIGA®), enzalutamide (XTANDI®), nilutamide (NILANDRON®), flutamide (EULEXIN®), bicalutamide (CASODEX®), orteronel (TAK-700, Tokai Pharmaceuticals, Inc.) Potent antiandrogens such as, for example, enzalutamide, abiraterone, ARN 509 (Aragon Pharmaceuticals, Inc.) and galeterone (TOK-001 or VN/124-1, Tokai Pharmaceuticals, Inc.), which are typically used in progressive, metastatic castration-resistant prostate cancer and which affect expression of a host of androgen-regulated molecules, such as PSMA expression.

Provided herein is a panel of biomarkers that were evaluated as predictors of efficacy with treatment, such as a PSMA targeted therapy, in castration-resistant metastatic prostate cancer. “Panel of biomarkers” is intended to refer to more than one biomarker that can be used to evaluate the likely responsiveness of a subject to a treatment as provided herein. “Likely responsiveness” refers to whether or not it would be expected that a treatment will have some benefit in a subject (or patient) as provided herein when administered. The likely responsiveness can be determined based on a score that is generated with any of the algorithms provided herein. The algorithms may be the correlation of expected outcome using one or more biomarker measurements as provided herein. Thus, the present invention, relates, at least in part to, a method of identifying and selecting prostate cancer patients likely to demonstrate efficacy of treatment using PSMA targeted therapy. The method in some embodiments relies upon the calculation of the level of a biomarker such as a neuroendocrine marker; a low level indicative of an efficacy response using, for example, PSMA ADC. In combination with low neuroendocrine levels, a PSA level of >100 ng/mL was also found to be predictive of efficacy using, for example, PSMA ADC.

Neuroendocrine markers as provided herein include serum neuroendocrine markers including chromogranin A (CgA) and neuron-specific enolase (NSE).

The present invention further provides an additional biomarker assay for identifying castration resistant metastatic prostate cancer subjects who are likely to benefit from PSMA targeted treatment. This biomarker assay comprises obtaining a relative or semi-quantitative measurement of PSMA density on PSMA+ circulating tumor cells (CTCs). Subjects having a high density of PSMA expression per PSMA+ CTCs are predicted to benefit from, for example, a PSMA ligand conjugate such as a PSMA ADC and may be selected for such a treatment accordingly.

In some aspects, diagnostic tests, methods, biomarker assays and kits, are provided that allow for the determination that a subject is likely to be responsive to a treatment. As used herein “treatment” refers to any therapy for treatment and can include a therapy that has not yet been administered to the subject or one that has been administered but may be continued based on the results of score of any of the assays, tests or methods provided herein. In some embodiments of any one of the methods, assays, kits or tests provided herein, the treatment comprises a PSMA targeted therapy for treating a PSMA expressing cancer. “PSMA targeted therapy” refers to an agent for treatment that is directed to PSMA expressing cells. Generally, such therapy is directed to PSMA expressing cells by way of ligands that bind, such as bind specifically to, PSMA. In some embodiments of any one of the methods, assays or tests provided herein, a step for providing or recommending a treatment to a subject or a treatment or materials describing a treatment are further comprised in the method, assay or test, respectively.

As used herein, a “PSMA ligand conjugate” comprises a molecule that binds specifically PSMA, such as an extracellular domain of PSMA, and is conjugated to a therapeutic agent. The therapeutic agent may be an anticancer agent. A “PSMA ligand,” therefore, herein refers to a molecule that specifically binds PSMA, as described herein. When a PSMA ligand is conjugated to an anticancer agent, the PSMA ligand conjugate is also referred to herein as a “PSMA ligand-anticancer agent conjugate”.

As used herein, “PSMA-expressing cells” refers to cells that express PSMA or that can express PSMA (e.g., human PSMA). PSMA is a 100 kD Type II membrane glycoprotein expressed in prostate tissues (Horoszewicz et al., 1987, Anticancer Res. 7:927-935; U.S. Pat. No. 5,162,504). PSMA was characterized as a type II transmembrane protein having sequence identity with the transferrin receptor (Israeli et al., 1994, Cancer Res. 54:1807-1811) and with NAALADase activity (Carter et al., 1996, Proc. Natl. Acad. Sci. U.S.A. 93:749-753). PSMA is expressed in increased amounts in prostate cancer (Horoszewicz et al., 1987, Anticancer Res. 7:927-935; Rochon et al., 1994, Prostate 25:219-223; Murphy et al., 1995, Prostate 26:164-168; and Murphy et al., 1995, Anticancer Res. 15:1473-1479). PSMA expression in cancerous prostate is approximately 10-fold greater than that in normal prostate. Expression in normal prostate is approximately 10-fold greater than that in the brain and is 50- to 100-fold greater than that of the liver or kidney. In most normal tissues, no expression of PSMA is observed.

Examples of PSMA ligands for use as provided herein include, without limitation, antibodies or antigen binding fragments thereof as well as small molecule ligands that bind specifically PSMA and may act as substrate mimics of enzymatic sites on PSMA. Antibodies that bind specifically to PSMA may be referred to herein as “PSMA antibodies.” Likewise, small molecule ligands that bind specifically PSMA may be referred to herein as “PSMA small molecule ligands.”

As used herein, “specific binding” refers to molecule (e.g., antibody) binding to a predetermined target (e.g., antigen), in this case PSMA (e.g., human PSMA). In some embodiments, that sequence of PSMA is set forth as SEQ ID NO: 1. Typically, the molecule binds with an affinity that is at least two-fold greater than its affinity for binding to a non-specific target (e.g., BSA, casein), which is a target other than PSMA, an isoform or variant of PSMA, or a closely-related target.

An antibody or an antigen-binding fragment thereof of a PSMA ligand conjugate may be any antibody or antigen-binding fragment thereof that binds PSMA (e.g., binds specifically to an epitope of PSMA). Examples of PSMA antibodies for use as provided herein include, without limitation, those listed provided in U.S. Pat. No. 8,114,965. Such antibodies or antigen-binding fragments thereof are incorporated herein by reference and include PSMA 3.7, PSMA 3.8, PSMA 3.9, PSMA 3.11, PSMA 5.4, PSMA 7.1, PSMA 7.3, PSMA 10.3, PSMA 1.8.3, PSMA A3.1.3, PSMA A3.3.1, 4.248.2, 4.360.3, 4.7.1, 4.4.1, 4.177.3, 4.16.1, 4.22.3, 4.28.3, 4.40.2, 4.48.3, 4.49.1, 4.209.3, 4.219.3, 4.288.1, 4.333.1, 4.54.1, 4.153.1, 4.232.3, 4.292.3, 4.304.1, 4.78.1 and 4.152.1, and antigen-binding fragments thereof.

In some embodiments, the antibody is produced by hybridomas referred to herein as PSMA 3.7 (PTA-3257), PSMA 3.8, PSMA 3.9 (PTA-3258), PSMA 3.11 (PTA-3269), PSMA 5.4 (PTA-3268), PSMA 7.1 (PTA-3292), PSMA 7.3 (PTA-3293), PSMA 10.3 (PTA 3247 PTA-3347), PSMA 1.8.3 (PTA-3906), PSMA A3.1.3 (PTA-3904), PSMA A3.3.1 (PTA-3905), Abgenix 4.248.2 (PTA-4427), Abgenix 4.360.3 (PTA-4428), Abgenix 4.7.1 (PTA-4429), Abgenix 4.4.1 (PTA-4556), Abgenix 4.177.3 (PTA-4557), Abgenix 4.16.1 (PTA-4357), Abgenix 4.22.3 (PTA-4358), Abgenix 4.28.3 (PTA-4359), Abgenix 4.40.2 (PTA-4360), Abgenix 4.48.3 (PTA-4361), Abgenix 4.49.1 (PTA-4362), Abgenix 4.209.3 (PTA-4365), Abgenix 4.219.3 (PTA-4366), Abgenix 4.288.1 (PTA-4367), Abgenix 4.333.1 (PTA-4368), Abgenix 4.54.1 (PTA-4363), Abgenix 4.153.1 (PTA-4388), Abgenix 4.232.3 (PTA-4389), Abgenix 4.292.3 (PTA-4390), Abgenix 4.304.1 (PTA-4391), Abgenix 4.78.1 (PTA-4652), and Abgenix 4.152.1 (PTA-4653), respectively.

These hybridomas were deposited pursuant to, and in satisfaction of, the requirements of the Budapest Treaty on the International Recognition of the Deposit of Microorganisms for the Purposes of Patent Procedure with the American Type Culture Collection (“ATCC”), having the address 10801 University Boulevard, Manassas, Va. 20110-2209, as an International Depository Authority.

In some embodiments, PSMA antibodies include the antibodies provided in U.S. Pat. Nos. 6,107,090, 6,649,163 and 6,962,981. Such antibodies are incorporated herein by reference. PSMA antibodies, therefore, include E99, J415, J533, and J591 monoclonal antibodies; monoclonal antibodies produced by hybridomas having ATCC Accession Numbers HB-12101, HB-12109, HB-12127 and HB-12126; and monoclonal antibodies produced by hybidomas having ATCC Accession Numbers HB12060 (3F5.4G6), HB12309 (3D7-1.1), HB12310 (4E10-1.14), HB12489 (1G3), HB12495 (1G9), HB12490 (2C7), HB12494 (3C4), HB12491 (3C6), HB12484 (3C9), HB12486 (3E6), HB12488 (3E11), HB12485 (3G6), HB12493 (4D4), HB12487 (4D8), HB12492 (4C8B9), HB12664 (3F6), HB12678 (2E4), HB12665 (3C2), HB12672 (2D4), HB12660 (4C8G8), HB12675 (2C4), HB12663 (4C11), HB12661 (1D11), HB12667 (4E8), HB12674 (2G5), HB12620 (4E6), HB12677 (1F4), HB12666 (2E3), HB12662 (3D8), HB12668 (4F8), HB12673 (3D2), HB12676 (1G7), HB12669 (3D4), HB12679 (5G10), and HB12671 (5E9). Antigen-binding fragments of these antibodies are also contemplated for use as PSMA ligands of the PSMA ligand conjugates provided herein.

As used herein, “antibody” refers to a glycoprotein comprising at least two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FRs). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

As used herein, “antigen-binding fragment” of an antibody refers to one or more portions of an antibody that retain the ability to bind specifically to an antigen (e.g., PSMA). The antigen-binding function of an antibody can be performed by fragments of a full-length antibody. Examples of binding fragments encompassed within the term “antigen-binding fragment” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, V and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional procedures, such as proteolytic fragmentation procedures, as described in J. Goding, Monoclonal Antibodies: Principles and Practice, pp 98-118 (N.Y. Academic Press 1983), which is hereby incorporated by reference, as well as by other techniques known to those with skill in the art. The fragments are screened for utility in the same manner as are intact antibodies.

As used herein, “isolated antibody” refers to an antibody that is substantially free of other antibodies having different antigenic specificities (e.g., an isolated antibody that specifically binds to PSMA is substantially free of antibodies that specifically bind antigens other than PSMA). An isolated antibody that specifically binds to an epitope, isoform or variant of PSMA may, however, in some embodiments, have cross-reactivity to other related antigens, e.g., from other species (e.g., PSMA species homologs). Moreover, an isolated antibody may, in some embodiments, be substantially free of other cellular material and/or chemicals.

Isolated antibodies of the invention encompass various antibody isotypes, such as IgG1, IgG2, IgG3, IgG4, IgM, IgA1, IgA2, IgAsec, IgD, IgE. As used herein, “isotype” refers to the antibody class (e.g., IgM or IgG1) that is encoded by heavy chain constant region genes. Antibodies can be full length or can include only an antigen-binding fragment such as the antibody constant and/or variable domain of IgG1, IgG2, IgG3, IgG4, IgM, IgA1, IgA2, IgAsec, IgD or IgE or could consist of a Fab fragment, a F(ab′)2 fragment, and a Fv fragment.

As used herein, “monoclonal antibody” refers to a preparation of antibody molecules of single molecular composition. A monoclonal antibody displays a single binding specificity and affinity for a particular epitope.

In some embodiments, an isolated antibody or antigen-binding fragment thereof may be selected for its ability to bind live cells expressing PSMA. In order to demonstrate binding of monoclonal antibodies to live cells expressing the PSMA, flow cytometry can be used.

In some embodiments, a PSMA antibody, or antigen-binding fragment thereof, binds to and is internalized with PSMA expressed on cells. Thus, a PSMA ligand conjugate comprising a PSMA antibody may be internalized with PSMA expressed on cells. The mechanism by which this internalization occurs is not critical to the practice of the present invention. For example, the antibody or antigen-binding fragment thereof can induce internalization of PSMA.

In some embodiments, a PSMA antibody, or antigen-binding fragment thereof, binds to a conformational epitope within the extracellular domain of the PSMA molecule. To determine if human PSMA antibodies bind to conformational epitopes, each antibody can be tested in assays using native protein (e.g., non-denaturing immunoprecipitation, flow cytometric analysis of cell surface binding) and denatured protein (e.g., Western blot, immunoprecipitation of denatured proteins). A comparison of the results will indicate whether the antibodies bind conformational epitopes. Antibodies that bind to native protein but not denatured protein are those antibodies that bind conformational epitopes, and are preferred antibodies in some embodiments.

In other embodiments, a PSMA antibody, or antigen-binding fragment thereof, binds to a dimer-specific epitope on PSMA. Generally, antibodies or antigen-binding fragments thereof that bind to a dimer-specific epitope preferentially bind the PSMA dimer rather than the PSMA monomer.

Other PSMA antibodies, or antigen-binding fragments thereof, provided herein include antibodies that bind specifically to an epitope on PSMA defined by a second antibody. To determine the epitope, one can use standard epitope mapping methods known in the art. For example, fragments (peptides) of PSMA antigen (preferably synthetic peptides) that bind the second antibody can be used to determine whether a candidate antibody binds the same epitope. For linear epitopes, overlapping peptides of a defined length (e.g., 8 or more amino acids) are synthesized. The peptides preferably are offset by 1 amino acid, such that a series of peptides covering every 8 amino acid fragment of the PSMA protein sequence are prepared. Fewer peptides can be prepared by using larger offsets, e.g., 2 or 3 amino acids. In addition, longer peptides (e.g., 9-, 10- or 11-mers) can be synthesized. Binding of peptides to antibodies can be determined using standard methodologies including surface plasmon resonance (e.g., BIACORE) and ELISA assays. For examination of conformational epitopes, larger PSMA fragments may be used as provided herein. Other methods that use mass spectrometry to define conformational epitopes have been described and may be used as provided herein (see, e.g., Baerga-Ortiz et al., Protein Science 11:1300-1308, 2002 and references cited therein). Still other methods for epitope determination are provided in standard laboratory reference works, such as Unit 6.8 (“Phage Display Selection and Analysis of B-cell Epitopes”) and Unit 9.8 (“Identification of Antigenic Determinants Using Synthetic Peptide Combinatorial Libraries”) of Current Protocols in Immunology, Coligan et al., eds., John Wiley & Sons. Epitopes can be confirmed by introducing point mutations or deletions into a known epitope, and then testing binding with one or more antibodies, or antigen-binding fragments thereof, to determine which mutations reduce binding of the antibodies, or antigen-binding fragments thereof.

In some embodiments, the PSMA antibody of a PSMA ligand conjugate is a monoclonal antibody that binds prostate specific membrane antigen (PSMA) protein dimer, PSMA protein dimer being a homodimer of PSMA protein monomer having the sequence of SEQ ID NO: 1, or an antigen-binding fragment thereof, wherein the antibody, or the antigen-binding fragment, (i) binds live cells and (ii) binds with at least a two-fold greater affinity to PSMA protein dimer than to PSMA protein monomer, as described in U.S. Pat. No. 8,114,965, incorporated by reference herein.

In some embodiments, PSMA antibodies are conjugated to radioactive molecules. An example of such a PSMA ligand conjugate, thus, includes 177Lu-J591, which contains monoclonal PSMA antibody J591 conjugated through 1,4,7,10-tetraazacyclododecane-1,4,7,10-tetraacetic acid (DOTA) to 177Lutetium (177Lu).

The PSMA ligand of a PSMA ligand conjugate may be any small molecule ligand that binds specifically PSMA. Such small molecule ligands may bind to the enzymatic site of PSMA in its native conformation. Also, such small molecule ligands may possess any one or more of the characteristics described above for PSMA antibody ligands.

In some embodiments, the small molecule ligand is based on a glutamate-urea-lysine heterodimer (e.g., glutamate-urea-lysine analog), or a glutamate-urea-glutamate based dimer, that binds specifically to an enzymatic site on PSMA. In some embodiments, such small molecule ligands are conjugated to a radionuclide as the anticancer, or cytotoxic, agent (e.g., cytotoxic radionuclide or radiotherapeutic isotope). Examples of PSMA ligand conjugates, thus, include glutamate-urea-amino acid based small molecule ligands conjugated to a radionuclide through an intervening linker such as 123I-MIP-1095 (also referred to as 123I-MIP-1466) and 123I-MIP-1072 (Molecular Insight Pharmaceuticals, Inc.). Other examples of PSMA small molecule ligands and PSMA ligand conjugates can be found in U.S. Pat. No. 8,465,725 and U.S. Pat. No. 8,487,129 and are incorporated herein by reference. In some embodiments, I123 may be substituted with other radiohalogens including those selected from the group consisting of I125, I131, I124, BR75, BR77 and F18.

The chemical structure of 123I-MIP-1095 (i.e., 123I—(S)-2-(3-((S)-1-carboxy-5-(3-(4-iodophenyl)ureido)pentyl)ureido)pentanedioic acid) is:

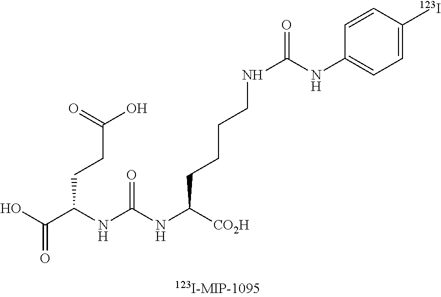 [SMILES: O=C(O)CC[C@H](NC(=O)N[C@@H](CCCCNC(=O)Nc1ccc([123I])cc1)C(=O)O)C(=O)O]

In another embodiment, the PSMA ligand conjugate is 124I-MIP-1095. In another embodiment, the PSMA ligand conjugate is 131I-MIP-1095.

The chemical structure of 123I-MIP-1072 (i.e., 123I—(S)-2-(3-((S)-1-carboxy-5-(4-iodobenzylamino)pentyl)ureido)pentanedioic acid) is:

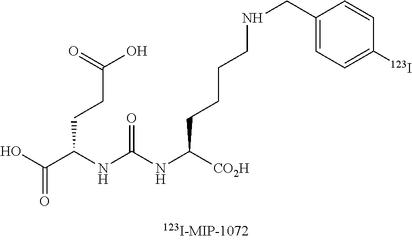 [SMILES: O=C(O)CC[C@H](NC(=O)N[C@@H](CCCCNCc1ccc([123I])cc1)C(=O)O)C(=O)O]

In some embodiments, the small molecule ligand of a PSMA ligand conjugate is a GL2 molecule as described in International Publication No. WO2010/005723. Any of the small molecules ligands provided herein, including a GL2 molecule, may be conjugated to a therapeutic agent by way of a nanoparticle (e.g., polymer-based, lipid-based and/or nucleic acid-based nanoparticles). In such embodiments, the nanoparticle may contain the therapeutic agent. Thus, in some embodiments, the PSMA ligand conjugate comprises a small molecule ligand conjugated to a nanoparticle that contains an anticancer or cytotoxic agent. Examples of such PSMA ligand conjugates include, without limitation, BIND-014 (Bind Biosciences, Inc.) described in International Publication No. WO2010/005723. The PSMA ligands and PSMA ligand conjugates of which are incorporated by reference herein.

PSMA small molecule ligands, in some embodiments, may be selected from the group consisting of compounds I, II, III and IV:

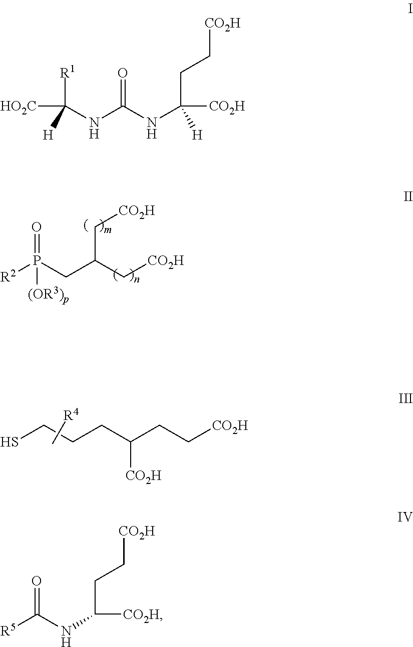

and enantiomers, stereoisomers, rotamers, tautomers, diastereomers, or racemates thereof; wherein m and n are each, independently, 0, 1, 2 or 3; p is 0 or 1; R1, R2, R4 and R5 are each, independently, selected from the group consisting of substituted or unsubstituted alkyl, substituted or unsubstituted aryl, and any combination thereof; and R3 is H or CH3; wherein R1, R2, R4 or R5 comprise a point of covalent attachment to the nanoparticle. For example, R1, R2, R4 and R5 may be each, independently, Ci1-6-alkyl or phenyl, or any combination of Ci1-6-alkyl or phenyl, which are independently substituted one or more times with OH, SH, NH2, or CO2H, and wherein the alkyl group may be interrupted by N(H), S or O. In some embodiments, for example, R1, R2, R4 and R5 are each, independently, CH2-Ph, (CH2)2-SH, CH2-SH, (CH2)2C(H)(NH2)CO2H, CH2C(H)(NH2)CO2H, CH(NH2)CH2CO2H, (CH2)2C(H)(SH)CO2H, CH2-N(H)-Ph, O—CH2-Ph, or O—(CH2)2-Ph, wherein each Ph may be independently substituted one or more times with OH, NH2, CO2H or SH.

PSMA small molecule ligands, in other embodiments, may be selected from the group consisting of:

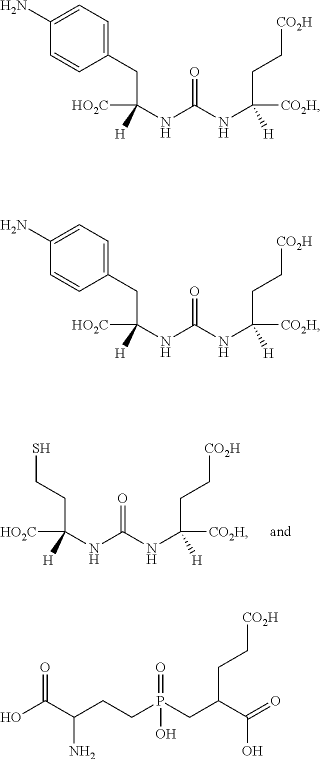 [SMILES: Nc1ccc(C[C@H](NC(=O)N[C@@H](CCC(=O)O)C(=O)O)C(=O)O)cc1 ; Nc1ccc(C[C@H](NC(=O)N[C@@H](CCC(=O)O)C(=O)O)C(=O)O)cc1 ; O=C(O)CC[C@H](NC(=O)N[C@@H](CCS)C(=O)O)C(=O)O ; "and" ; NC(CCP(=O)(O)CC(CCC(=O)O)C(=O)O)C(=O)O]

and enantiomers, stereoisomers, rotamers, tautomers, diastereomers, or racemates thereof; and wherein the NH2, OH or SH groups serve as a point of covalent attachment, or may be selected from the group consisting of

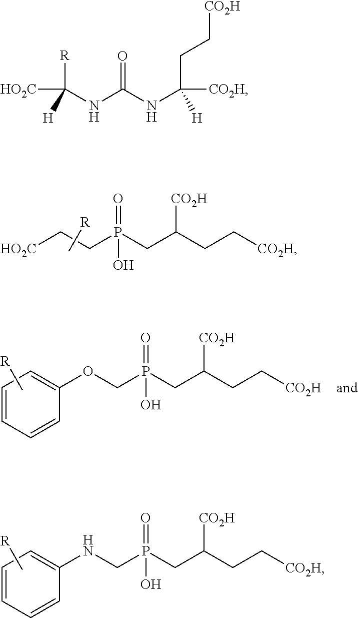

and enantiomers, stereoisomers, rotamers, tautomers, diastereomers, or racemates thereof; wherein R is independently selected from the group consisting of NH2, SH, OH, CO2H, Ci_{—}6-alkyl that is substituted with NH2, SH, OH or CO2H, and phenyl that is substituted with NH2, SH, OH or CO2H, and wherein R serves as the point of covalent attachment.

PSMA small molecule ligands, in yet other embodiments, may be selected from the group consisting of:

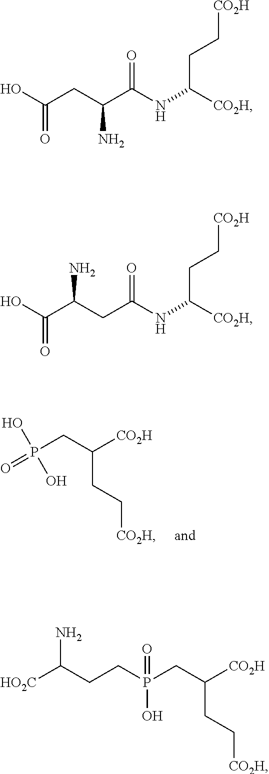

and enantiomers, stereoisomers, rotamers, tautomers, diastereomers, or racemates thereof; any of which may be further substituted with NH2, SH, OH, CO2H, Ci1-6-alkyl that is substituted with NH2, SH, OH or CO2H, or phenyl that is substituted with NH2, SH, OH or CO2H, wherein these functional groups serve as the point of covalent attachment. For example, a low-molecular weight PSMA ligand may be

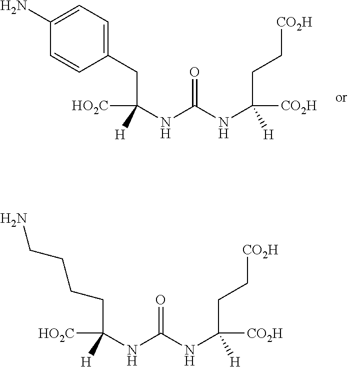 [SMILES: Nc1ccc(C[C@H](NC(=O)N[C@@H](CCC(=O)O)C(=O)O)C(=O)O)cc1 ; NCCCC[C@H](NC(=O)N[C@@H](CCC(=O)O)C(=O)O)C(=O)O]

and enantiomers, stereoisomers, rotamers, tautomers, diastereomers, or racemates thereof; wherein the NH2 groups serve as the point of covalent attachment. Attachment may be to a linker, polymer, particle, etc.

In some embodiments, the PSMA small molecule ligand that comprises a molecule that is or mimics a substrate that binds the enzymatic site on PSMA includes 2-[3-(1,3-dicarboxypropyl)ureido]pentanedioic acid (DUPA). In some embodiments, such small molecule ligands are conjugated to a chemotherapeutic agent, such as tubulysin hydrazide (TubH). The synthesis and uses of an example of such a PSMA ligand conjugate (EC1069) are described in Kularatne, S A et al J Med Chem 2010, 53, 7767-7777; Kularatne, S A et al Mol Pharmaceutics Vol 6, no 3, 780-789, 2009. EC1719 is another example of a PSMA ligand conjugate that includes TubH. EC1069 and EC1719 can target the chemotherapy drug to PSMA receptors expressed on prostate cancer cells (Endocyte). Other EC1069 and EC1719 analogs linking DUPA and TubH can also target PSMA receptors expressed on prostate cancer cells. Thus, also contemplated herein are analogs of EC1069 and analogs of EC1719. The terms “EC1069” and “EC1719,” therefore, encompasses EC1069, EC1719 and analogs thereof. The linkers of the analogs, in some embodiments, may be peptides with D-amino-acid(s), or peptides attached with sugar moieties, amides or esters. An example of a linker, therefore, is D-γ-Glu D-Asp-D-Phe-D-Cys. Other linkers may be used as provided herein.

Conjugation of one or more therapeutic agents to a PSMA ligand can include many chemical mechanisms, for instance covalent binding, affinity binding, intercalation, coordinate binding, electrostatic binding and complexation. Conjugation may also include encapsulation and is intended to refer to any mechanism by which one component may be associated with another component. Conjugation may be direct conjugation of the therapeutic agent to the PSMA ligand or it may be indirect, such as via a linker, polymer, particle etc., and it is the linker, polymer, particle, etc. to which the therapeutic agent is bound.

Covalent binding can be achieved either by direct condensation of existing side chains or by the incorporation of external bridging molecules. Many bivalent or polyvalent agents are useful in coupling protein molecules to other proteins, peptides or amine functions, etc. For example, the literature is replete with coupling agents such as carbodiimides, diisocyanates, glutaraldehyde, diazobenzenes, and hexamethylene diamines. This list is not intended to be exhaustive of the various coupling agents known in the art but, rather, is exemplary of the more common coupling agents.

In some embodiments, wherein the PSMA ligand is an antibody, it is contemplated the antibody is first derivatized, and then the therapeutic agent is attached to the derivatized product. Suitable cross-linking agents for use in this manner include, for example, SPDP (N-succinimidyl-3-(2-pyridyldithio)propionate), and SMPT, 4-succinimidyl-oxycarbonyl-methyl-(2-pyridyldithio)toluene.

In some embodiments, where the agent is a protein toxin, it may be fused to the PSMA ligand by genetic methods to form a hybrid immunotoxin fusion protein. The fusion proteins can include additional peptide sequences, such as peptide spacers that operatively attach, for example, the PSMA ligand and toxin, as long as such additional sequences do not appreciably affect the targeting or toxin activities of the fusion protein. The proteins, in some embodiments, may be attached by a peptide linker or spacer, such as a glycine-serine spacer peptide, or a peptide hinge, as is well known in the art. Thus, for example, if the PSMA ligand is a PSMA antibody, the C-terminus of PSMA antibody can be fused to the N-terminus of the protein toxin molecule to form an immunotoxin that retains the binding properties of the PSMA antibody. Other fusion arrangements will be known to one of ordinary skill in the art.

Examples of anticancer agents for use as provided herein include, without limitation, cytotoxic agents, chemotherapeutic agents and agents that act on tumor neovasculature.

Cytotoxic agents include, but are not limited to, cytotoxic radionuclides, chemical toxins and protein toxins. Cytotoxic radionuclides or radiotherapeutic isotopes include alpha-emitting isotopes such as, for example, 225Ac, 211At, 212Bi, 213Bi, 212Pb, 224Ra, 223Ra. Cytotoxic radionuclides or radiotherapeutic isotopes include beta-emitting isotopes such as, for example, 186Rh, 188Rh, 177Lu, 90Y, 131I, 67Cu, 64Cu, 153Sm, 166Ho. In some instances, cytotoxic radionuclides may emit Auger and/or low energy electrons and include the isotopes 123I, 124I, 125I, 131I, 75Br, 77Br and 18F.

Radionuclides typically are coupled to an antibody or antigen-binding fragment thereof by chelation. For example, in the case of metallic radionuclides, a bifunctional chelator is commonly used to link the isotope to the antibody or other protein of interest. Typically, the chelator is first attached to the antibody, and the chelator-antibody conjugate is contacted with the metallic radioisotope. A number of bifunctional chelators have been developed for this purpose, including the diethylenetriamine pentaacetic acid (DTPA) series of amino acids described in U.S. Pat. Nos. 5,124,471, 5,286,850 and 5,434,287, which are incorporated herein by reference. As another example, hydroxamic acid-based bifunctional chelating agents are described in U.S. Pat. No. 5,756,825, the contents of which are incorporated herein. Another example is the chelating agent termed p-SCN-Bz-HEHA (1,4,7,10,13,16-hexaazacyclo-octadecane-N,N′,N″,N′″,N″″,N′″″-hexaacetic acid) (Deal et al., J. Med. Chem. 42:2988, 1999), which is an effective chelator of radiometals such as 225Ac. Yet another example is DOTA (1,4,7,10-tetraazacyclododecane N,N′,N″,N′″-tetraacetic acid), which is a bifunctional chelating agent (see McDevitt et al., Science 294:1537-1540, 2001) that can be used in a two-step method for labeling followed by conjugation.

Chemical toxins or chemotherapeutic agents include, but are not limited to, members of the enediyne family of molecules, such as calicheamicin and esperamicin. Chemical toxins or chemotherapeutic agents can also include pyrrolobenzodiazepine (PBD) dimers (e.g., SJG-136, SG2000, SG2202, SG2285 as described in Hartley J A et al., Cancer Res. 2010, 70(17):6849-58), calicheamicins, colchicine, ispinesib (a novel small molecule inhibitor of kinesin spindle protein), combrestatin (e.g., combrestatin A4), maytansine derivatives such as maytansinoid DM4 (N2′-deacetyl-N2′-(4-mercapto-4-methyl-1-oxopentyl)maytansine) and maytansinoid DM1 (mertansine), methotrexate, doxorubicin, melphalan, chlorambucil, ARA-C, vindesine, mitomycin C, cis-platinum, etoposide, bleomycin and/or 5-fluorouracil. Other antineoplastic agents include dolastatins (U.S. Pat. Nos. 6,034,065 and 6,239,104) and derivatives thereof. Dolastatins and derivatives thereof include dolastatin 10 (dolavaline-valine-dolaisoleuine-dolaproine-dolaphenine) and the derivatives auristatin PHE (dolavaline-valine-dolaisoleuine-dolaproine-phenylalanine-methyl ester) (Pettit, G. R. et al., Anticancer Drug Des. 13(4):243-277, 1998; Woyke, T. et al., Antimicrob. Agents Chemother. 45(12):3580-3584, 2001), aurastatin E (e.g., monomethylauristatin norephedrine), aurastatin F (e.g., monomethylauristatin phenylalanine) and the like. Toxins also include poisonous lectins, plant toxins such as ricin, abrin, modeccin, botulina and diphtheria toxins. Other chemotherapeutic agents are known to those skilled in the art and may be used as provided herein.

Agents that act on the tumor vasculature include, but are not limited to, tubulin-binding agents (e.g., anti-tubulin agents) such as tubulysin and derivatives thereof (Kaur et al., Biochem J. 396(Pt 2):235-242, 2006), combrestatin A4 (Griggs et al., Lancet Oncol. 2:82, 2001), angiostatin and endostatin (reviewed in Rosen, Oncologist 5:20, 2000, incorporated by reference herein) and interferon inducible protein 10 (U.S. Pat. No. 5,994,292). A number of other antiangiogenic agents are also contemplated and include: 2ME2, angiostatin, angiozyme, anti-VEGF RhuMAb, Apra (CT-2584), avicine, benefin, BMS275291, carboxyamidotriazole, CC4047, CC5013, CC7085, CDC801, CGP-41251 (PKC 412), CM101, combretastatin A-4 prodrug, EMD 121974, endostatin, flavopiridol, genistein (GCP), IM-862, ImmTher, interferon alpha, interleukin-12, gefitinib (ZD1839), marimastat, metastat (Col-3), neovastat, octreotide, paclitaxel, penicillamine, photofrin, photopoint, PI-88, prinomastat (AG-3340), PTK787 (ZK22584), RO317453, solimastat, squalamine, SU 101, SU 5416, SU-6668, suradista (FCE 26644), suramin (metaret), tetrathiomolybdate, thalidomide, TNP-470 and vitaxin. Additional antiangiogenic agents are described by Kerbel, J. Clin. Oncol. 19(18s):45s-51s, 2001, which is incorporated by reference herein. Such agents are contemplated for use in the PSMA ligand conjugates provided herein.

In some embodiments, a PSMA ligand conjugate is a PSMA antibody-drug conjugate. Non-limiting examples of PSMA antibody-drug conjugates are described in US-2007-0160617-A1 and US-2011-0250216-A, such examples of each of which are incorporated by reference herein. In some embodiments, a PSMA antibody-drug conjugate comprises an antibody or antigen-binding fragment thereof that specifically binds PSMA and is conjugated to a dolastatin 10 derivative, in particular auristatins such as, MMAE (also referred to herein as monomethylauristatin E or monomethylauristatin norephedrine) or MMAF (also referred to herein as monomethylauristatin F or monomethylauristatin phenylalanine).

MMAE or MMAF can be conjugated to an antibody or antigen-binding fragment thereof using methods known to those of ordinary skill in the art (e.g., See, Niemeyer, C M, Bioconjugation Protocols, Strategies and Methods, Humana Press, 2004) or as described herein. In some embodiments, more than one MMAE or MMAF molecule is conjugated to the antibody or antigen-binding fragment thereof. In other embodiments, 1, 2, 3, 4, 5, 6, 7 or 8 MMAE or MMAF molecules are conjugated to the antibody or antigen-binding fragment thereof. In still other embodiments, at least 2, 3, 4 or 5 MMAE or MMAF molecules are conjugated to the antibody or antigen-binding fragment thereof. In further embodiments, 2, 3, 4 or 5 MMAE or MMAF molecules are conjugated to the antibody or antigen-binding fragment thereof.

In some embodiments, the PSMA ligand conjugate is PSMA antibody (or antigen-binding fragment thereof)-maleimide caproyl-valine-citrulline-p-aminobenzyloxycarbonyl-monomethylauristatin norephedrine, PSMA antibody (or antigen-binding fragment thereof)-maleimide caproyl-valine-citrulline-p-aminobenzylcarbamate-monomethylauristatin norephedrine, PSMA antibody (or antigen-binding fragment thereof)-maleimide caproyl-monomethylauristatin norephedrine, PSMA antibody (or antigen-binding fragment thereof)-maleimide caproyl-valine-citrulline-p-aminobenzyloxycarbonyl-monomethylauristatin phenylalanine, PSMA antibody (or antigen-binding fragment thereof)-maleimide caproyl-valine-citrulline-p-aminobenzylcarbamate-monomethylauristatin phenylalanine or PSMA antibody (or antigen-binding fragment thereof)-maleimide caproyl-monomethylauristatin phenylalanine. In any of the foregoing, the PSMA antibody or antigen-binding fragment thereof may be any of the antibodies or antigen-binding fragments provided herein.

A composition, in some embodiments, includes a physiologically or pharmaceutically acceptable carrier, excipient, or stabilizer. As used herein, “pharmaceutically acceptable carrier” or “physiologically acceptable carrier” includes any and all salts, solvents, dispersion media, coatings, antibacterial and antifungal agents, isotonic and absorption delaying agents, and the like that are physiologically compatible. A “pharmaceutically-acceptable carrier,” as used herein, refers to one or more compatible solid or liquid fillers, diluents or encapsulating substances that are suitable for administration into a human. The term “carrier” denotes an organic or inorganic ingredient, natural or synthetic, with which the active ingredient is combined to facilitate the application. A carrier may be suitable for intravenous, intramuscular, subcutaneous, parenteral, spinal or epidermal administration (e.g., by injection or infusion).

In some embodiments, a composition may be administered to a subject in pharmaceutically-acceptable amounts and in pharmaceutically-acceptable compositions. The term “pharmaceutically acceptable” means a non-toxic material that does not interfere with the effectiveness of the biological activity of the active ingredients (e.g., PSMA ligands, anticancer agents,). Such compositions may contain salts, buffering agents, preservatives, compatible carriers, and optionally other therapeutic agents, such as supplementary immune potentiating agents including adjuvants, chemokines and cytokines. When used in medicine, the salts should be pharmaceutically acceptable, but non-pharmaceutically acceptable salts may conveniently be used to prepare pharmaceutically-acceptable salts thereof and are not excluded.

A salt retains the desired biological activity of the parent compound and does not impart any undesired toxicological effects (see e.g., Berge, S. M., et al. (1977) J. Pharm. Sci. 66: 1-19). Examples of such salts include acid addition salts and base addition salts.

The pharmaceutical compositions may contain suitable buffering agents, including: acetic acid in a salt; citric acid in a salt; boric acid in a salt; and phosphoric acid in a salt.

In some embodiments, a composition may contain suitable preservatives, such as: benzalkonium chloride; chlorobutanol; parabens and/or thimerosal.

In some embodiments, a composition may conveniently be presented in unit dosage form and may be prepared by any of the methods well-known in the art of pharmacy. All methods include the step of bringing the active compound(s) (e.g., PSMA ligand, anticancer agent) into association with a carrier that constitutes one or more accessory ingredients. In some embodiments, compositions are prepared by uniformly and intimately bringing the active compound into association with a liquid carrier, a finely divided solid carrier, or both, and then, if necessary, shaping the product.

Compositions suitable for parenteral administration conveniently comprise a sterile aqueous or non-aqueous preparation of PSMA ligand conjugates, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. Carrier formulations suitable for oral, subcutaneous, intravenous, intramuscular, etc. administration can be found in Remington's Pharmaceutical Sciences, Mack Publishing Co., Easton, Pa. Any of the compositions provided herein may be sterile.

Active compounds (e.g., PSMA ligand, anticancer agent) can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

A composition can be administered by any conventional route, including injection or by gradual infusion over time. The administration may, for example, be oral, intravenous, intraperitoneal, intramuscular, intracavity, intratumor, or transdermal. When compositions are used therapeutically, preferred routes of administration include intravenous administration.

Compositions as provided herein, in some embodiments, may be administered in effective amounts. An “effective amount” is that amount of an active compound (e.g., PSMA ligand conjugate) that alone, or together with further doses, produces the desired response, e.g., inhibits cell proliferation of PSMA-expressing cells and/or kills PSMA-expressing cells. For cancer, this may involve only slowing the progression of a cancer, for example, temporarily, although more preferably, it involves halting the progression of the cancer permanently. This can be monitored by routine methods. The desired response to treatment of cancer or other disease or condition also can be delaying the onset or even preventing the onset of the cancer or other disease or condition.

Such effective amounts will depend, of course, on the particular condition being treated (e.g., PSMA-expressing cancer), the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient/subject may insist upon a lower dose or tolerable dose for medical reasons, psychological reasons or for virtually any other reason.

Compositions as provided herein, in some embodiments, are sterile and contain an effective amount of PSMA ligand conjugates, etc. for producing the desired response in a unit of weight or volume suitable for administration to a patient/subject. The response can, for example, be measured by determining the physiological effects of the composition, such as regression of a tumor or decrease of disease symptoms. Other assays will be known to one of ordinary skill in the art and can be employed for measuring the level of the response.

The doses of compositions administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

For example, doses of PSMA ligand conjugate can range from about 10 μg/kg to about 100,000 μg/kg. Based on the composition, the dose can be delivered continuously, such as by continuous pump, or at periodic intervals. Desired time intervals of multiple doses of a particular composition can be determined without undue experimentation by one skilled in the art. Other protocols for the administration of compositions will be known to one of ordinary skill in the art, in which the dose amount, schedule of administration, sites of administration, mode of administration and the like vary from the foregoing.

In some embodiments, the dose of PSMA ligand conjugate is administered intravenously. In such embodiments, the dose of PSMA ligand conjugate may be about 1.0 mg/kg to 2.5 mg/kg. In some embodiments, the dose of PSMA ligand conjugate may be 1.0 mg/kg to 2.5 mg/kg. For example, in some embodiments, the dose of PSMA ligand conjugate administered intravenously is 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, or 2.5 mg/kg. In some embodiments, the dose of PSMA ligand administered intravenously conjugate is about 1.0 mg/kg to 2.3 mg/kg. In some embodiments, the dose of PSMA ligand administered intravenously conjugate is 1.0 mg/kg to 2.3 mg/kg. In an embodiment, the PSMA ligand conjugate is a PSMA ADC, and the PSMA ADC is provided in a dose of about 1.8 mg/kg to 2.3 mg/kg. In an embodiment, the PSMA ligand conjugate is a PSMA ADC, and the PSMA ADC is provided in a dose of 1.8 mg/kg to 2.3 mg/kg.

The length of time during which a PSMA ligand conjugate is administered administered intravenously may vary. In some embodiments, a PSMA ligand conjugate may be administered intravenously for 30 minutes, 35 minutes, 40 minutes, 45 minutes, 50 minutes, 55 minutes, 60 minutes, 65 minutes, 70 minutes, 75 minutes, 80 minutes, 85 minutes or 90 minutes.

In some embodiments, a PSMA ligand conjugate is administered intravenously at repeated intervals such as, for example, once a week, once every two weeks, or once every three weeks for up to a total of four, six or eight doses. In some embodiments, the PSMA ligand conjugate is administered intravenously twice a week, or more.

In some embodiments, a PSMA ligand conjugate may be administered intravenously for about 60 minutes once every three weeks at a dose of about 1.0 mg/kg to 2.3 mg/kg for up to a total of eight doses.

In general, doses of radionuclides delivered by a PSMA ligand conjugate provided herein can range from about 0.01 mCi/Kg to about 10 mCi/kg. In some embodiments, the dose of radionuclide ranges from about 0.1 mCi/Kg to about 1.0 mCi/kg. In some embodiments, the dose of radionuclide ranges from 0.1 mCi/Kg to 1.0 mCi/kg. In one embodiment, the PSMA ligand conjugate is 131I-MIP-1095 provided in an I.V. dose of about 1 to 10 GBq. In one embodiment, the PSMA ligand conjugate is 131I-MIP-1095 provided in an I.V. dose of 1 to 10 GBq. In another embodiment, 131I-MIP-1095 is provided in a dose range of 2 to 8 GBq. In yet another embodiment, the mean dose is about 5 GBq. In yet another embodiment, the mean dose is 5 GBq. The optimal dose of a given isotope can be determined empirically by simple routine titration experiments well known to one of ordinary skill in the art.

Administration of compositions provided herein to mammals other than humans, e.g. for testing purposes or veterinary therapeutic purposes, is carried out under substantially the same conditions as described above.

Compositions (e.g., that comprise PSMA ligand conjugate) as provided herein have in vitro and in vivo diagnostic and therapeutic utilities. For example, these compounds can be administered to cells in culture, e.g., in vitro or ex vivo, or in a subject, e.g., in vivo, to treat, prevent or diagnose cancer or other disease or condition. As used herein, the term “subject” is intended to include humans and non-human animals. Preferred subjects include a human patient having a disorder characterized by expression, typically aberrant expression (e.g., overexpression) of PSMA.

The compositions provided herein, in some embodiments, may be used in conjunction with other therapeutic treatment modalities. Such other treatments include surgery, radiation, cryosurgery, thermotherapy, hormone treatment, chemotherapy, vaccines, and other immunotherapies.

Subjects with prostate cancer, in some embodiments, have undergone, are undergoing, or will undergo hormone therapy. Thus, in some embodiments, the compositions provided herein may be administered to a subject subsequent to, together with, or prior to hormone therapy, such as for prostate cancer. Examples of hormone therapies for prostate cancer include, without limitation: luteinizing hormone-releasing hormone agonists (e.g., leuprolide, goserelin, and buserelin), which can stop the testicles from making testosterone; antiandrogens (e.g., flutamide, bicalutamide, enzalutamide and nilutamide), as discussed elsewhere herein, which can block the action of androgens such as testosterone; drugs that can prevent the adrenal glands from making androgens (e.g., ketoconazole and aminoglutethimide); orchiectomy, which is a surgical procedure to remove one or both testicles, the main source of male hormones such as testosterone, to decrease the amount of hormone being produced; and estrogens, which can prevent the testicles from making testosterone.

A myriad of subjects may benefit from the methods and compositions provided herein. In some embodiments, such a subject has progressive metastatic castration-resistant prostate cancer despite a castrate level of serum testosterone (e.g., <50 mg/dL) and having had prior chemotherapy with docetaxel. In other embodiments, the subject has metastatic castration resistant prostate cancer, has had prior treatment with taxane chemotherapy and has received and progressed on abiraterone and/or enzalutamide. In yet other embodiments, the subject has progressive metastatic castration-resistant prostate cancer despite a castrate level of serum testosterone, has had prior treatment with abiraterone and/or enzalutamide, and has had no prior treatment with cytotoxic chemotherapy. In still other embodiments, the subject has progressive metastatic castration-resistant prostate cancer despite a castrate level of serum testosterone and has had one prior treatment with abiraterone and/or enzalutamide. In further embodiments, the subject has progressive metastatic castration-resistant prostate cancer despite a castrate level of serum testosterone and has had no prior treatment with abiraterone and or enzalutamide. In additional embodiments, the subject has asymptomatic or minimally symptomatic metastatic castration-resistant prostate cancer despite a castrate level of serum testosterone and has had no prior treatment with abiraterone and/or enzalutamide. In some embodiments, the subject has stable metastatic castration-resistant prostate cancer and is receiving treatment with abiraterone and/or enzalutamide. In other embodiments, the subject has biochemically recurrent prostate cancer and has previously undergone a primary therapy (e.g., radical prostatectomy (e.g., open, laparoscopic, or robot-assisted) or radiation therapy (e.g., dose-escalated three-dimensional conformal RT, intensity-modulated RT, brachytherapy, or a combination thereof)). In yet other embodiments, the subject has localized high-risk prostate cancer (e.g., prostate specific antigen (PSA) greater than 10 nanogram per milliliter (ng/ml); PSA velocity greater than 2 ng/ml per/year (defined as a rise in PSA of greater than 2 ng/ml in the preceding 12 month period); Gleason score greater than or equal to 7 (4+3); or Gleason score 6 if either PSA greater than or equal to 10 ng/ml or PSA velocity greater than or equal to 2 ng/ml/year) and is a candidate for prostatectomy.

Also provided herein are kits comprising the composition(s). In some embodiments, the kits comprise a container containing biomarker assay reagents as described elsewhere herein or a therapeutic such as a PSMA ligand conjugate (or the components thereof) or both the assay reagents and PSMA ligand conjugate (or the components thereof). The kits can further contain at least one additional reagent as provided herein. In some embodiments, a kit may comprise a carrier being compartmentalized to receive in close confinement therein one or more containers or series of containers such as test tubes, vials, flasks, bottles, syringes, or the like. One container or series of containers may contain one or more assay reagents. Another container, or series of containers in some embodiments, may contain a PSMA ligand conjugate (or the components thereof). The components of the kits can be packaged either in aqueous medium or in lyophilized form. The components of the conjugates can be supplied either in fully conjugated form, in the form of intermediates or as separate moieties to be conjugated by the user of the kit.

Kits may, in some embodiments, also comprise a diluent and/or instructions for reconstituting lyophilized forms, or instructions for diluting aqueous components of the kits. Kits may also comprise instructions for using the biomarker assay reagents and/or selecting the subjects or patients for a treatment modality as provided elsewhere herein.

In some embodiments, the described techniques may be implemented as a software tool. The tool may be implemented in any suitable manner and may be executed by one or more processors at one or more servers so that it is accessed by users over a network. For example, the tool may receive from a user input values and provide the results of an analysis to the user. In one embodiment, the analysis comprises an algorithm as provided herein, such as an algorithm for assigning a high or low value based on one or more biomarkers as provided herein. The results may be provided to the user in any suitable manner—for example, saved in a file of a suitable format and sent to a user via a suitable communication medium. Additionally or alternatively, the results may be displayed on a display of a computing device. The tool may be configured to receive user input relating to any parameters used by the tool.

In other embodiments, the software tool implementing the described techniques may be downloaded to a user's computer or otherwise obtained by the user. In such embodiments, the tool may be executed on the user's computer.

Such alterations, modifications, and improvements are intended to be part of this disclosure, and are intended to be within the spirit and scope of the invention. Accordingly, the foregoing description and drawings are by way of example only.

The above-described embodiments of the present invention can be implemented in any of numerous ways. For example, the embodiments may be implemented using hardware, software or a combination thereof. When implemented in software, the software code can be executed on any suitable processor or collection of processors, whether provided in a single computer or distributed among multiple computers. Such processors may be implemented as integrated circuits, with one or more processors in an integrated circuit component. Though, a processor may be implemented using circuitry in any suitable format.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks. In some embodiments, a suitable cloud computing technology may be utilized to implement the described techniques.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the invention may be embodied as a computer readable storage medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other tangible computer storage medium) encoded with one or more programs that, when executed on one or more computers or other processors, perform methods that implement the various embodiments of the invention discussed above. As is apparent from the foregoing examples, a computer readable storage medium may retain information for a sufficient time to provide computer-executable instructions in a non-transitory form. Such a computer readable storage medium or media can be transportable, such that the program or programs stored thereon can be loaded onto one or more different computers or other processors to implement various aspects of the present invention as discussed above. As used herein, the term “computer-readable storage medium” encompasses only a computer-readable medium that can be considered to be a manufacture (i.e., article of manufacture) or a machine. Alternatively or additionally, the invention may be embodied as a computer readable medium other than a computer-readable storage medium, such as a propagating signal.

The terms “program” or “software” are used herein in a generic sense to refer to any type of computer code or set of computer-executable instructions that can be employed to program a computer or other processor to implement various aspects of the present invention as discussed above. Additionally, it should be appreciated that according to one aspect of this embodiment, one or more computer programs that when executed perform methods of the present invention need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags or other mechanisms that establish relationship between data elements.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference. However, the citation of any reference is not intended to be admission that said reference is prior art.

### EXAMPLES

### Example 1

### Assessment of the Anti-Tumor Activity of PSMA ADC

A Phase 2, open-label, multicenter study was used to assess the anti-tumor activity and tolerability of prostate specific membrane antigen (PSMA) antibody-drug conjugate (ADC) in two groups of subjects with metastatic castration-resistant prostate cancer (mCRPC). One group was comprised of approximately 75 subjects who must have received at least one taxane-containing chemotherapy regimen (e.g. docetaxel, cabazitaxel). If a subject had received more than two cytotoxic chemotherapy regimens, Sponsor approval was required for study participation. The second group is comprised of approximately 35 subjects who are cytotoxic chemotherapy-naïve. Subjects who are cytotoxic chemotherapy-naïve must have received and progressed on, be ineligible for, refused, have an intolerance to, or not have access to Radium-223. Both groups of subjects must also have received and progressed on abiraterone acetate and/or enzalutamide. If a subject is unable to receive abiraterone acetate and/or enzalutamide, Sponsor approval is required for participation in the study.

PSMA ADC 2.3 mg/kg was administered as an intravenous (IV) infusion over approximately 60 minutes once every three weeks (Q3W) for up to eight doses (unless dose delay or dose reduction is required). Subjects who were dosed prior to a specific date continued to receive PSMA ADC 2.5 mg/kg if it is well tolerated, or it was reduced to a dose of 2.3 mg/kg at the principle investigator's (PI's) discretion. The subject was be weighed prior to each cycle and dosing was calculated on a mg/kg basis prior to each dose. The dose for subjects weighing greater than 100 kg was calculated based on a weight of 100 kg.

Dose delays and/or reductions within the scope of the titration guidelines do not require Sponsor approval; however it is recommended that the clinical research associate (CRA) is contacted regarding either dose delay or dose reduction. Dose reductions were in steps of 0.2 mg/kg. Dosing was not less than 1.9 mg/kg nor more than the starting dose of 2.3 mg/kg for newly enrolled subjects or 2.5 mg/kg for subjects who were dosed prior to a certain date.

### Study Conduct:

### Screening and Treatment Periods

Subjects enter into a screening period (up to three weeks), during which inclusion/exclusion criteria was to be assessed and eligibility determined. During the study, the following assessments were to be performed:

### Radiologic Imaging

Screening imaging measurements were performed once it has been determined that a subject met all other inclusion/exclusion criteria and prior to first dose (cycle 1, day 1). However, subjects who have had the appropriate imaging performed within 30 days prior to first dose used these images for screening measurements. Following screening measurements, the imaging will be performed at cycle 5 and at end of study (EOS), unless an earlier assessment is clinically indicated. The imaging techniques used at screening was used throughout the study.
The preferred imaging modality was IV contrast enhanced computerized tomography (CT) scan of chest, upper and lower abdomen and pelvis.
Subjects who had a contraindication to IV CT contrast material had a contrast enhanced magnetic resonance imaging (MRI) of the upper and lower abdomen and pelvis and a non-contrast CT of the chest.
In the event a subject presented with both a contraindication to contrast enhanced CT and MRI, then a non-contrast CT scan of chest, upper and lower abdomen and pelvis was performed.
   All radiological images were to be sent to BioClinica, the central imaging reader.

### Radiologic Image Data Conclusion

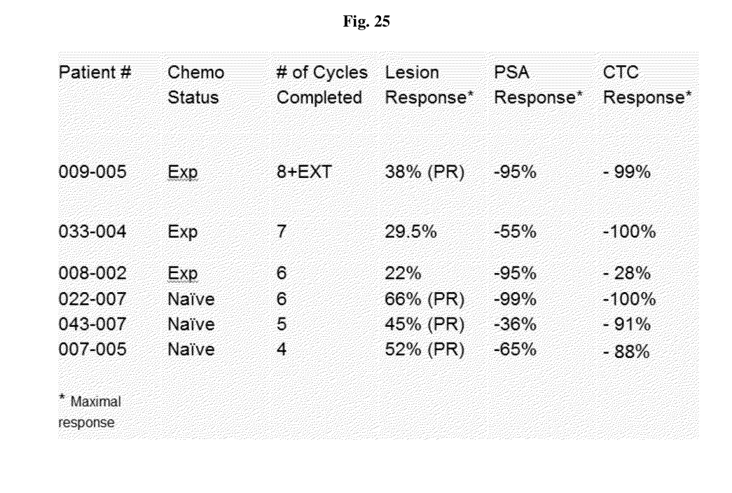

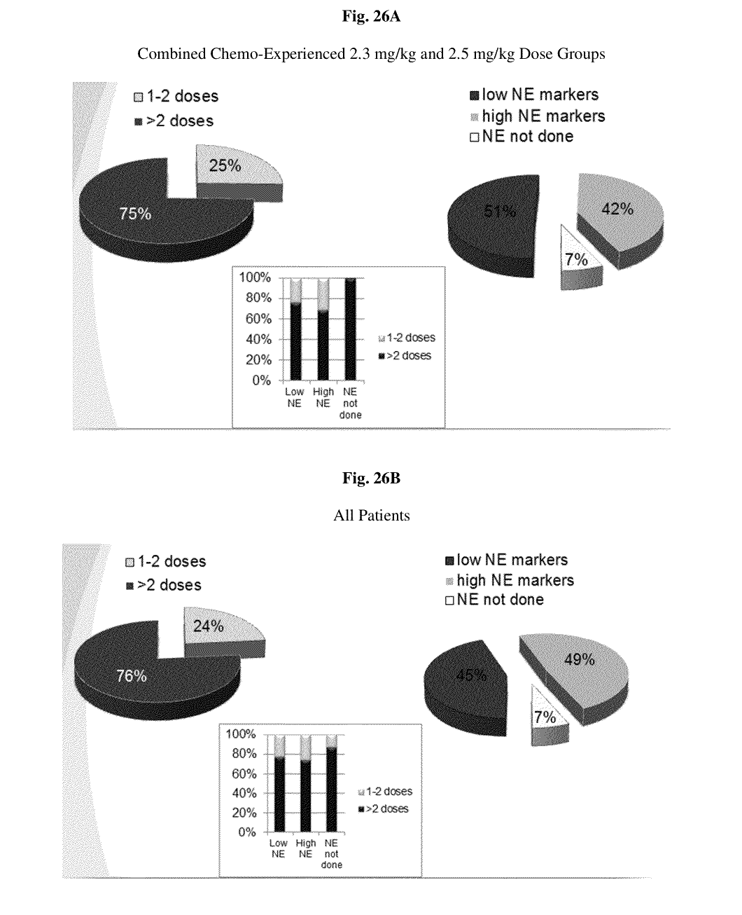

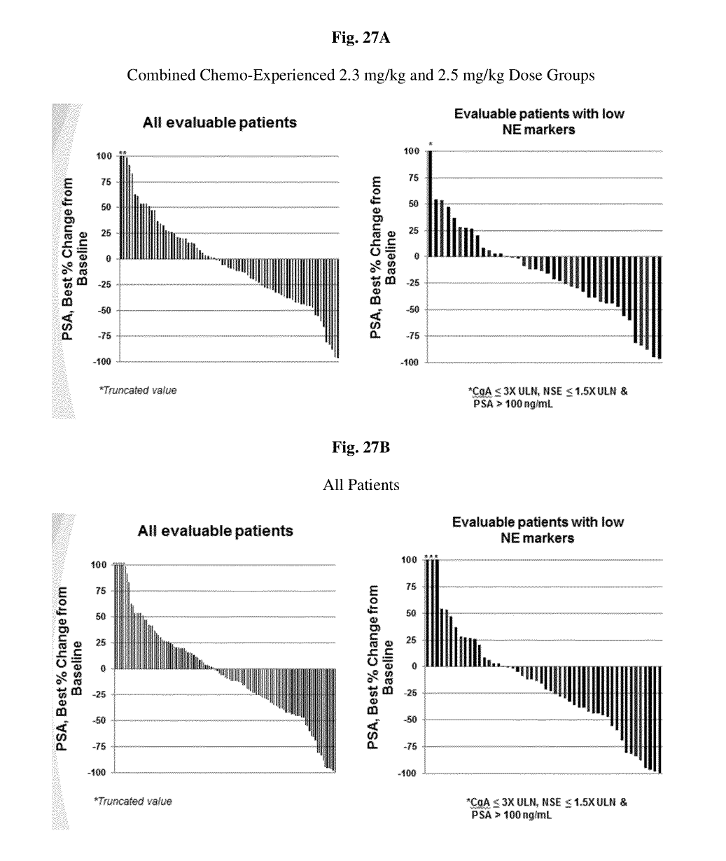

Administration of PSMA ADC at 2.5 mg·kg or 2.3 mg/kg in the highly pre-treated mCRPC patient population resulted in biochemical and radiologic responses. The radiologic response was consistent with PSA and CTC responses. Thirty one of 105 patients had measurable target lesions (chemotherapy-experience plus chemo-naïve). In patients with measurable target lesions (31 of 105 patients), 74% achieved stable disease (SD), 13% achieved Partial Response (PR), and 13% showed Progressive Disease (PD) after treatment with PSMA ADC. FIG. 24 shows a graph of best Response Evaluation Criteria In Solid Tumors (RECIST) target lesion change from baseline following treatment with PSMA ADC. FIG. 25 shows a summary of radiologic response in patients with measurable target lesions correlated with PSA and CTC response.

### Laboratory Samples for Biomarker Analysis

PSMA expression was evaluated on available tumor tissue by immunohistochemistry (IHC) and on circulating tumor cells (CTC) by immunofluorescence
PSA was obtained at screening and prior to each dose in the study and at EOS.
Serum testosterone was obtained at screening
CTC was obtained at screening and prior to dosing at cycles 1, 2, 3, 4, and at EOS
Two separate PSMA CXC samples were obtained at baseline to determine PSMA expressing CTCs (cycle 1, day 1) and to determine the magnitude of PSMA expression (density) using Method 1 and Method 2.
Serum for analysis of chromogranin A (CgA) and neuron-specific enolase (NSE) was collected at baseline only (cycle 1, day 1).

### Inclusion Criteria

In order to be eligible for the study, subjects must have met all of the following inclusion criteria:
1. Males, age ≧18 years.
2. <150 kg
3. Eastern Cooperative Oncology Group (ECOG) performance status of 0, 1, or 2
4. Life expectancy ≧six months.
5. Must have histologically or cytologically confirmed prostate adenocarcinoma.
6. mCRPC as determined by the results of imaging studies
7. A castrate level of serum testosterone (<50 ng/dL) at screening (testosterone was measured by the central laboratory by mass spectrometry if colorimetric assay is greater than 50).
8. Prior and/or ongoing androgen-deprivation therapy consisting of either orchiectomy or luteinizing hormone-releasing hormone (LHRH) agonists, with or without an anti-androgen. If chemically castrated, subjects must have agreed to stay on LHRH agonist medication for the duration of the study.
9. a) Prior history of treatment with at least one taxane-containing chemotherapy regimen (e.g. docetaxel, cabazitaxel). If a subject received more than two cytotoxic chemotherapy regimens, Sponsor approval was required for study participation.

### OR

b) Subjects with no prior history of treatment with a cytotoxic chemotherapy regimen.

10. Must have received and progressed on abiraterone acetate and/or enzalutamide and must have waited a minimum of 30 days from their last dose of abiraterone acetate and/or enzalutamide prior to receiving their first dose of PSMA ADC. If subject is unable to receive abiraterone acetate and/or enzalutamide, Sponsor approval was required for participation in the study.
11. Were willing and able to provide written informed consent and written authorization for use and release of health and research information.

### Exclusion Criteria

Subjects meeting the following exclusion criteria are not eligible for this study:
1. Predominant histologically or cytologically confirmed neuroendocrine prostate cancer.
Duration of Treatment: 8 cycles (each cycle lasted three weeks)

### Treatment Therapy, Dosage Form/Regimen, and Route of Administration:

### Study

| Study | Dosage | Dosage and | |
|---|---|---|---|
| Drug | Form | Administration | Dosing Frequency |
| PSMA | Injection | *IV infusion | One IV infusion every three weeks |
| ADC | | administered | (eight doses): |
| | | over | Cycles 1, 2, 3, 4, 5, 6, 7 and 8 |
| | | approximately | |
| | | 60 minutes | |

*The subject was to be weighed prior to each cycle and dosing was to be calculated on a mg/kg basis prior to each dose. The dose for subjects weighing greater than 100 kg was calculated based on a weight of 100 kg.

### Endpoints:

### Response/Duration

PSA responses as described by the Prostate Cancer Clinical Trials Working Group (PCWG2) criteria (57)

Percentage of change in total serum PSA from baseline to 12 weeks

Maximum percent decline in total serum PSA that occurs at any point after baseline In addition, assessments of PSA response as follows:

Responders with a decrease in total serum PSA≧25%
over the first 12 weeks of treatment
over the study (approximately 24 weeks)
maximal decline at any time during the study

Responders with a decrease in total serum PSA≧50%
over the first 12 weeks of treatment
over the study (approximately 24 weeks)
maximal decline at any time during the study

Changes in total serum PSA from baseline
Change from baseline total serum PSA
Percent change from baseline total serum PSA

### PSA Doubling Time (PSADT)

All PSA values used in the calculation of PSADT should be ≧0.20 ng/mL and following a rising trend. All PSA values should be obtained using the same assay, preferably at the same laboratory and collected at approximately the same time of day.

Minimum requirements for the calculation are three total serum PSA values obtained over three months with a minimum of four weeks between measurements.

Tumor responses in bone, visceral or nodal metastases according to the Revised Response Evaluation Criteria (RECIST 1.1) (56).

Objective (tumor) response in Target Lesions (number and proportion of subjects)
Complete response (CR)
Partial Response (PR)
Progressive Disease (PD)
Stable Disease (SD)
Not Evaluable (NE)

Response Evaluation Criteria in Non-Target Lesions (number and proportion of subjects)
Complete Response (CR)
Non-Complete Response (Non-CR)/Non-Progressive Disease (Non-PD)
Progressive Disease (PD)
Not Evaluable (NE)
   Change from baseline in circulating tumor cells (CTCs)
Proportion of subjects who achieve CTC<5 cells
   Time to response
Total serum PSA
Tumor measurements
   Time to disease progression
Total serum PSA
Tumor measurements
   Overall survival (OS; months)
   Progression free survival (PFS; months)
   Duration of anti-tumor response (months)
Total serum PSA
Tumor measurements

### Skeletal Related Events (SREs)

Time to occurrence of SREs
Frequency of new SREs
Proportions of subjects with a new SRE

### Symptomatic Reponses and Patient Reported Outcomes

Tumor-associated symptoms
Two baseline assessments will be obtained prior to the first cycle of study drug and will be used as the reference point for questionnaires administered prior to each subsequent dose.
Pain scores will be evaluated using the BPI-SF.
Fatigue scores will be evaluated using the BFI.

Analgesic consumption will be monitored throughout the study.

Quality of life will be evaluated using the FACT-P.

### Example 2

### Biomarker Assays

### Neuroendocrine Assays

### Chromogranin A (CgA) Assay

A patient blood sample was collected prior to initiation of treatment (baseline serum CgA) into a red-top tube, lavender-top (EDTA) tube or green-top (heparin) tube and the serum or plasma separated. The CgA level was determined using a radioimmunoassay (LabCorp, Test No. 140848). The assay reference interval is 0-5 nmol/L (ULN=5 nmole/L; equivalent to 245 ng/mL) is based on a population of normal subjects. (O'Connor D T, Pandlan M R, Carlton E et al, Radioimmunoassay of Chromagranin A: In vitro Stability, Exploration of the Neuroendocrine Character of Neoplasia, and Assessment of the Effects of Organ Failure, Clin Chem, 1989, 35(8):1631-7).

### Neuron-Specific Enolase (NSE) Assay

A patient blood sample was collected prior to initiation of treatment (baseline NSE) into a red-top tube and the serum separated and the NSE level determined using an Enzyme immunoassay (EIA) (LabCorp, Test No. 140624). The assay reference interval is 0 to 12.5 ng/mL (ULN=12.5 ng/mL) is based on a population of normal subjects. (Kaiser E, Kuzmits R, Pregant P et al, “Clinical Biochemistry of Neuron Specific Enolase”, Clin Chim Acta, 1989, 183 (1):13-31; Virji M A, Mercer D W, Herberman R B, “Tumor Markers in Cancer Diagnosis and Prognosis,” Cancer J Clin, 1988, 38(2):104-26).

### Prostate-Specific Antigen (PSA) Assay

A patient blood sample was collected prior to initiation of treatment (baseline PSA) and at each dosing cycle/day 1 (8 cycles) into a red-top tube and the serum separated and the PSA level determined using electrochemiluminescence immunoassay (ECLIA) (LabCorp, Test No. 010322; Mohler J, Bahnson, R R, Boston, B et al, NCCN Clinical Practice Guidelines in Oncology: Prostate Cancer, J Natl Compr Canc Netw, 2010, 8(2):162-200.

### Circulating Tumor Cell Assay

The assay provides enumeration of circulating tumor cells (CTC) of epithelial origin in whole blood. Whole blood was collected prior to initiation of treatment (baseline CTC) and at Cycle 2/day 1, Cycle 3/Day 1, Cycle 4/Day 1 and at the end of the study into a CellSave preservative tube and the specimen analysed by immunomagnetic selection, identification and enumeration of CTC in peripheral blood sample enrichment. Cells were classified as CTCs if they were cytokeratin-FITC positive, DAPI positive and CD45-APC negative (LabCorp, Test No. 502088; de Bono J S, Sher H I, Montgomery R B et al. Circulating tumor cells predict survival benefit from treatment in metastatic castration-resistant prostate cancer. Clin. Cancer Res. 2008; 14 (19):6302-6309).

### PSMA⁺ Circulating Tumor Cell Assay

### Method 1

An anti-PSMA antibody (PSMA 3.9, a murine antibody; PSMA Development Co., ATCC PTA-3258) labeled with phycoerythrin (m3.9-PE) was used to stain PSMA⁺ CTC cells isolated from whole blood samples collected from multiple donors in CellSave tubes (CTCs in 7.5 mL blood). The samples were prepared for staining and analysis using a CellTrack® AutoPrep® System. A CellTracks Analyzer II (Veridex) was then used to analyze the CTC samples. The optimal concentration of PSMA antibody was 2 μg/mL to achieve maximal signal with minimal background. An exposure time of 0.5 s was used. Further optimization experiments confirmed that binding by the m3.9-PE conjugate was not significantly affected by the presence of PSMA-ADC. The number of total CTCs, the percent positive PSMA CTCs and the mean fluorescent intensity of PSMA⁺ CTCs were determined.

Based on the experiments described above, the following assay protocol was developed.

### Blood Collection

1. Collect whole blood aseptically by venipuncture or from a venous port into CellSave Preservative tube. At least 7.5 mls of blood is needed to run the assay.

2. Invert the tube several times to prevent clotting.

3. Once blood is collected in a CellSave tube, it is stable for up to 96 h.

### Dilution of the m3.9-PE Antibody

1. Dilute the stock antibody concentration to 28.3 μg/ml in PBS, 0.1% sodium azide, and 0.3% bovine serum albumin.

2. Perform a second 2.5× dilution to 11.4 μg/ml in PBS, 0.1% sodium azide, and 0.3% bovine serum albumin.

3. After performing the second dilution, add the appropriate amount of this concentration of antibody to the tumor phenotyping reagent cup according to the number of samples to be processed. The final concentration of the antibody after addition to the − reaction mixture is 2 μg/ml.

### Sample Processing

1. Remove the CellSearch CXC kit and CXC control kit (if needed) from the refrigerator 30 mins prior to use and allow to warm to room temperature.

2. Label each 15 ml AutoPrep conical tube for each patient sample in the batch, including name or study number, date, and medical record on tubes (if applicable).

3. Transfer 7.5 ml of whole blood from the CellSave tube to the corresponding labeled conical tube.

4. Add 6.5 ml of dilution buffer (supplied by Veridex) to each conical tube containing 7.5 ml whole blood.

5. Use supplied caps to seal conical tubes, and mix blood and dilution buffer evenly by gently inverting five times.

6. Centrifuge tubes at 800×g for 10 min with the brake off. Perform centrifugation at room temperature.

7. Process the sample with the CellTracks Autoprep system within 1 hour of sample preparation.

8. When prompted to select a reagent kit, choose the CellSearch CXC Kit.

9. When prompted to select a Marker Reagent, choose “User Defined”, if a marker reagent is to be included in the run.

10. Add the appropriate volume of diluted m3.9-PE antibody to the tumor phenotyping reagent cup according to the number of samples that will be processed with the reagent.

11. Place the reagent cup in Position 1 on the reagent carrier.

12. Continue to follow the on-screen prompts on the CellTracks Autoprep system to process the samples.

### Control Preparation (CXC Control Kit)

1. Label one 15 ml Autoprep conical with lot-specific orange bar code label. One control is required to be run as part of a batch every 24 h.

2. Vortex the control vial gently for 5 s.

3. Gently mix the control vial by inversion 5 times.

4. Transfer contents of vial to labeled conical tube.

5. Do not spin control with samples to be run on the AutoPrep, but it should be run in a batch with the samples.

6. When loading samples for analysis, include control as the final sample in the run.

### Scanning on the CellTracks Analyzer II

1. After processing, unload samples in MagNest and lay flat in the dark for at least 20 minutes before scanning.

2. While waiting (or beforehand), turn the Analyzer on and allow the mercury lamp to warm up for 15 mins.

3. Place the system verification cartridge on the MagNest holder.

4. Select QC.Test, Verification, and then Start.

5. Once the scanning is complete, the Analyzer will display Pass or Fail.

6. If the verification passes, the test samples can now be scanned.

7. Scan control and patient samples on the Analyzer II using an exposure time of 0.5 s in the PE channel.

8. After successful scanning, turn off the analyzer.

The performance of the PSMA staining assay was also verified on clinical samples, whole blood samples collected from 19 metastatic prostate cancer patients attained from a commercial vendor.

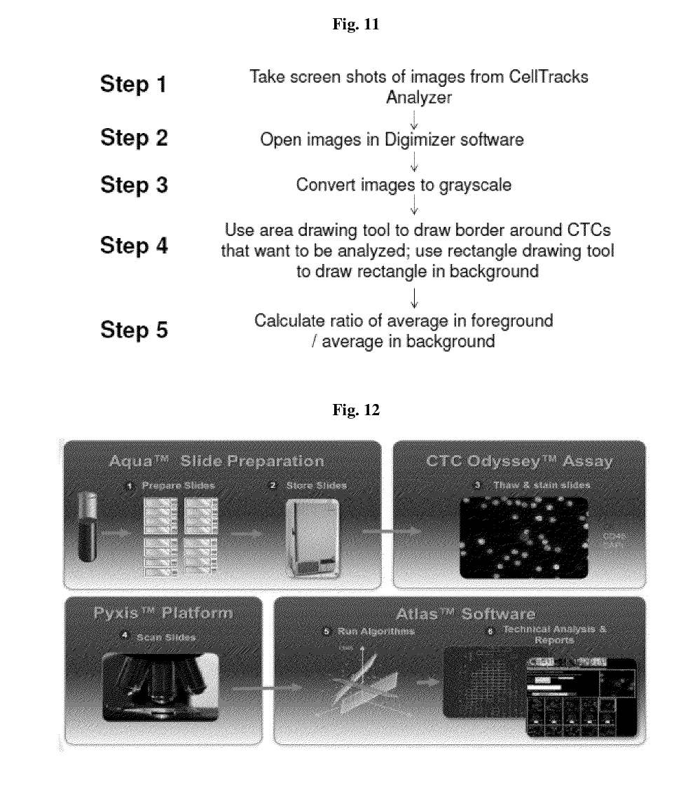

Digimizer software (MedCalc Software bvba, Ostend, Belgium) was used to score PSMA expression or density levels on CTCs. The Digimizer workflow is presented schematically in FIG. 11. For example, a picture was taken of CTC images displayed on the CellTracks Analyzer II from LNCaP cells isolated from blood. The image was opened in the Digimizer software and analyzed according to the workflow presented in FIG. 11. A single value for PSMA expression was determined for each CTC by calculating the ratio of average intensity of foreground to average intensity of background. In an alternative preferred embodiment, a single value for PSMA expression was determined for each CTC by calculating (foreground-background)×100.

It was found that captured CTCs were amendable to quantitative PSMA immunofluorescence using the Digimizer software package. It was apparent that the expression level of PSMA on CTCs is extremely heterogenous. This method may be valuable in predicting which patients may respond best to PSMA ADC therapy based on PSMA expression levels.

### Method 2

The PSMA CTC test was developed using LNCaP (ATCC CRL-1740; high cell surface PSMA expression), 22Rv1 (ATCC CRL-2505; low cell surface PSMA expression) and PC3 (no PSMA expression) cells spiked into normal donor blood. Nucleated blood cells (approximately 1 mL volume) were plated onto glass slides and stored in a −80° C. biorepository. The slides were subjected to immunofluorescent staining followed by CTC identification using the Pyxis™ Scanning Platform. The four-color assay evaluated PSMA expression on individual CTCs, identified as cells which are cytokeratin+, CD45−, and with an intact DAPI+ nucleus. Multiple anti-PSMA antibody clones were evaluated over a range of assay conditions and antibody concentrations to identify the optimal clone and assay conditions that would result in high specificity and sensitivity. Acceptance criteria included signal intensities in LNCaP v. PC3, subcellular localization of PSMA, and potential interference by a PSMA targeted therapy. Clinical feasibility of the optimized assay was assessed on samples from 20 CRPC patients who were either naïve or resistant to taxane-based chemotherapy. A schematic depicting the workflow for CTC collection and detection is presented in FIG. 12 and includes (1) placing nucleated cells from blood samples onto slides (approximately 1 mL of blood sample per slide), (2) storing the slides in a −80 C biorepository, (3) staining the slides to detect CK, CD45, DAPI and PSMA, (4) scanning the slides, (5) running multi-parametric digital pathology algorithms and (6) confirming, by both software and human readers, CTCs and quantitation of biomarker expression.

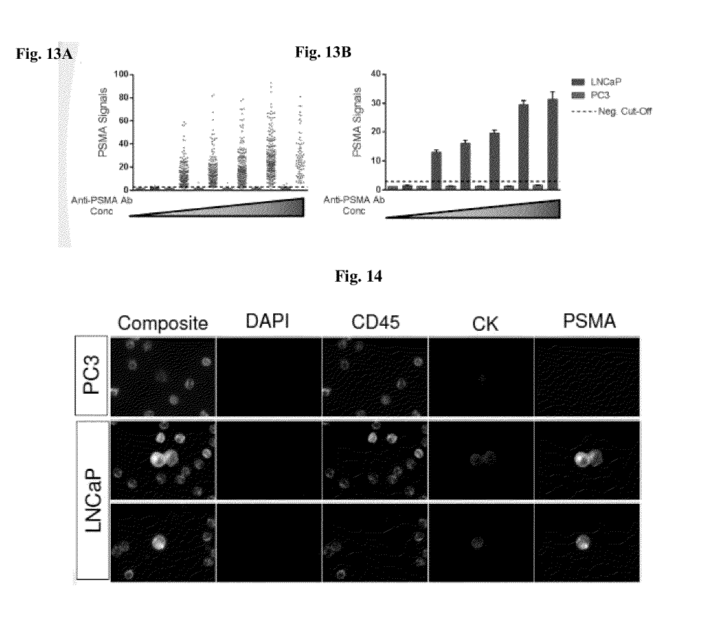

The rabbit monoclonal anti-PSMA antibody (Abcam, clone EPR6253, ab133579) showed a clear separation between the high PSMA-expressing cell line (LNCaP) and the negative cell line (PC3). An antibody titration curve with the rabbit monoclonal is shown in FIG. 13. Increasing concentrations of an anti-PSMA rabbit monoclonal antibody were applied to either LNCaP (high PSMA, dark gray) or PC3 (no PSMA, light gray) cells to generate a titration curve. The scattered plot (FIG. 13A) shows PSMA signals measured on each cell whereas the bar graph (FIG. 13B) shows mean PSMA signals and the standard error of the mean (SEM). At the optimal antibody concentration (denoted by an *), the average PSMA signal measured in LNCaP cells was 20-fold higher than that in PC3 cells. The negative cutoff was set at 3, below which a cell is considered “negative” for PSMA. Representative images of the PSMA staining of PC3 (no PSMA) cells and LNCaP (high PSMA) are shown in FIG. 14. The PSMA CTC Assay detected a predominantly membrane-localized staining pattern for PSMA, which is distinct from the cytoplasmic staining pattern of CK, the epithelial cell marker.

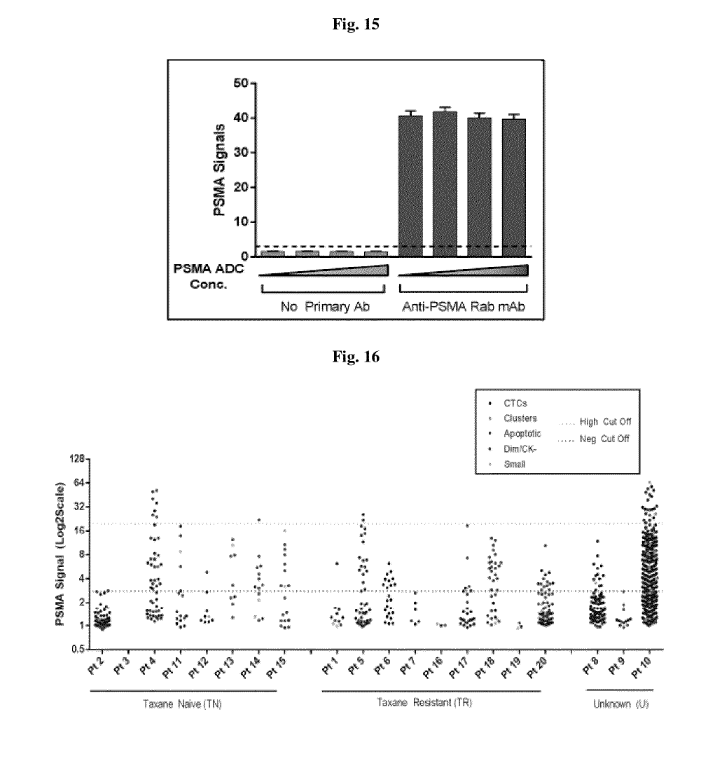

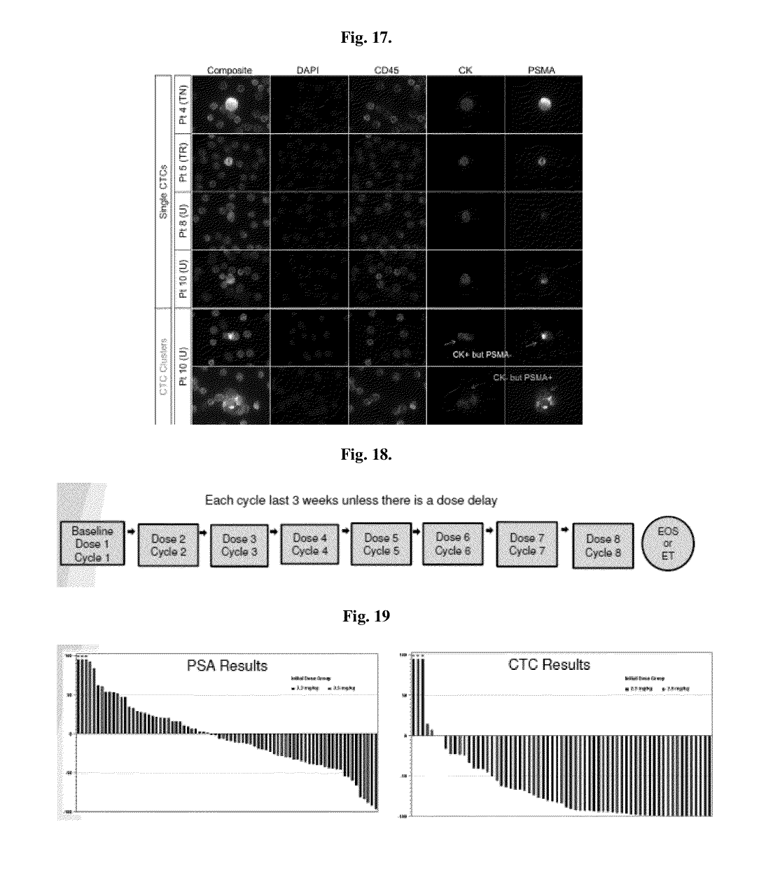

The acceptable criteria for an anti-PSMA antibody were met with the rabbit monoclonal anti-PSMA antibody. The PSMA CTC assay using this antibody resulted in an average PSMA signal intensity for the high PSMA-expressing cell line LNCaP that was 20-fold higher than that for the PC3 cell line, 18-fold higher than the no primary antibody control, and 10-fold higher than the minimum cutoff. Detection with this antibody demonstrated a predominantly membrane-localized pattern of staining for PSMA that was distinct from cytokeratin. To ensure that the antibody did not interact with PSMA ADC, a PSMA ADC interaction assay was performed as presented in FIG. 15. LNCaP cells were first treated with either vehicle control (first bar in each group) or with increasing concentrations of PSMA ADC. The cells were subsequently spiked into white blood cells isolated from a healthy donor. The resulting samples were subjected to the PSMA CTC assay to detect PSMA. Bars on the left represent results from no primary antibody controls; bars on the right represent results from the PSMA CTC assay. Assay performance was unaffected by the presence of PSMA ADC, a PSMA targeted antibody-drug conjugate that is in phase 2 clinical testing.

Clinical feasibility of the optimized assay was assessed on samples from 20 CRPC patients independently sourced. 8 of the patients were naïve to taxane-based chemotherapy, 9 were resistant, and 3 had unknown taxane-based chemotherapy histories. The assay demonstrated utility in detecting and quantitating PSMA expression on individual and clustered CTCs as well as on apoptotic, cytokeratin-negative, and small cytokeratin-positive CTC subpopulations. CTCs and CTC subpopulations were detected in all but one patient. The results and exemplary images from the clinical feasibility study are presented in FIGS. 12 and 13, respectively, and summarized in Table 1.

PSMA expression was successfully detected and quantitated on diverse types of circulating cells present in the blood of patients with CRPC. Assay performance was unaffected by the presence of a PSMA targeted therapeutic agent. PSMA CTC data are being collected in the ongoing phase 2 study of PSMA ADC for comparison with treatment outcomes. Development of a PSMA CTC assay may help enable patient selection for anti-PSMA therapeutics and real time monitoring of the disease using CTCs as a fluid biopsy.

TABLE 1
Summary table of PSMA CTC assay clinical feasibility data

| Patient ID | Taxane status | CTCs/mL | % PSMA+ |
|---|---|---|---|
| 10 | Unknown | 485 | 60% |
| 4 | Naïve | 52 | 58% |
| 14 | Naïve | 2 | 50% |
| 6 | Resistant | 29 | 50% |
| 5 | Resistant | 80 | 44% |
| 20 | Resistant | 45 | 17% |
| 11 | Naïve | 4 | 14% |
| 1 | Resistant | 8 | 14% |
| 8 | Unknown | 147 | 13% |
| 17 | Resistant | 25 | 4% |
| 2 | Naïve | 131 | 0% |
| 3 | Naïve | 0 | 0% |
| 12 | Naïve | 6 | 0% |
| 13 | Naïve | 1 | 0% |
| 15 | Naïve | 3 | 0% |
| 7 | Resistant | 7 | 0% |
| 16 | Resistant | 2 | 0% |
| 18 | Resistant | 3 | 0% |
| 19 | Resistant | 2 | 0% |
| 9 | Unknown | 15 | 0% |

### PSMA⁺ Immunohistochemistry (IHC) Assay

### Collection, Processing, and Shipment of Samples for IHC Samples

Either existing biopsy samples and/or new biopsy samples available as embedded blocks or fixed slides are sent to LabCorp. Biopsy samples may be from a primary tumor or from a metastasis. The embedded block(s) measure 2¾ cm wide by 3 cm long. Two fixed slides are prepared for the IHC samples.

Once informed consent has been obtained, the shipment of the existing biopsy and/or new biopsy samples for IHC staining and/or RNA/DNA is completed.

### Reagent Provided

The anti-PSMA antibody is provided in liquid form as tissue culture supernatant (containing fetal bovine serum) dialyzed against 0.05 mol/L Tris-HCL, pH 7.2, and 0.015 mol/L sodium azide. The anti-PSMA reagent contains stabilizing protein.

Clone 3E6 Isotype: IgG1, kappa

Mouse IgG concentration mg/L. See label on vial.

The anti-PSMA antibody is used at a dilution of 1:100 when performing IHC using the EnVision™ detection system. Optimal antibody concentrations may vary depending on specimen and preparation method.

### Reagent Specificity

Monoclonal mouse anti-PSMA has been demonstrated to react in Western blotting with PSMA from LNCaP cell lysate, seminal fluid and with recombinant baculovirus expressed PSMA. Clone 3E6 also binds a 100 kD protein in LNCaP lysates, corresponding to PSM’. Clone 3E6 was also found to react with PSMA on LNCaP cells by flow cytometry. Monoclonal anti-PSMA clone 3E6 recognizes an epitope present in the 57-134 amino acid region of the extracellular portion of the PSMA molecule, as determined by Western blot analysis of baculovirus expressed PSMA fragments.

### Specimen Preparation

Anti-PSMA antibodies can be used on formalin-fixed, paraffin-embedded tissue sections. Pretreatment of tissues with proteolytic enzymes is generally not recommended.

The deparaffinized tissue sections are treated with heat prior to the IHC staining procedure. Heat-induced epitope retrieval (HIER) involves immersion of tissue sections in a pre-heated buffer solution and maintaining heat in a water bath (95-99° C.). Alternative heat sources may be used for HIER upon validation against the procedure. Use a 20-minute heating protocol for HIER performed at 95-99° C.; after thermal treatment, the jar is allowed, with buffer and slides, to cool for 20 minutes at room temperature. Rinsing with buffer or deionized water is performed following HIER. For greater adherence of tissue sections to glass slides, the use of Silanized Slides (DakoCytomation, code S3003) is generally recommended. Target Retrieval Solution pH 9.0 (DakoCytomation, code S2368) or 10× Concentrate (DakoCytomation, code S2367) is generally recommended.

The formalin-fixed paraffin embedded (FFPE) tissue IHC staining method used a PSMA antibody, PSMA 3E6 (Dako), and was established in a previous LabCorp Clinical Trials validation study. An indirect IHC procedure was performed using a MACH4 Universal HRP-Polymer Detection Kit (Biocare Medical). The secondary detection antibody was Nemesis Mouse Probe.

### Example 3

### Clinical Results

### Interim Analysis

75 patients were treated in a phase 2 trial to test the anti-tumor activity and tolerability of PSMA ADC in taxane-refractory mCRPC. Patients with progressive mCRPC following taxane treatment and a score of 0, 1, or 2 on the Eastern Cooperative Oncology Group Performance Scale (ECOG PS) were eligible for the trial. Patients that had previously undergone treatment with >2 cytotoxic chemotherapies were excluded from the trial.

35 patients initiated treatment at 2.5 mg/kg. Due to neutropenia, the remaining 35 patients began at 2.3 mg/kg. Demographics and baseline characteristics of the patient population and treatment are summarized in Table 2. FIG. 22 provides the percentage of patients that received various prior treatments. 41% had also received cabazitaxel. Adverse events (AEs) were consistent with those seen in phase 1; most common significant AEs were neutropenia (grade 4, 6.7% and 11.4% at 2.3 and 2.5 mg/kg, respectively and peripheral neuropathy (grade≧equal to 3, 6.7% (2.3 mg/kg) and 5.7% (2.5 mg/kg)). Two patients at 2.5 mg/kg died of sepsis associated with neutropenia. 47% of patients at 2.3 mg/kg and 53% of patients at 2.5 mg/kg had declines in CTC from ≧5 to <5 cells/7.5 ml blood and 57.1% (2.3 mg/kg) and 74.1% (2.5 mg/kg) had ≧50% CTC declines. 26.1% (2.3 mg/kg) and 16.1% (2.5 mg/kg) had PSA declines of ≧30%. 15.56% (2.3 mg/kg) and 6.45% (2.5 mg/kg) had PSA declines of ≧50%. PSA and CTC responses to treatment were associated with higher PSMA expression on CTCs and lower neuroendocrine (NE) markers (NSE and CgA) at baseline. The CTC conversion rate (≧5 CTC at baseline to <5 CTC following treatment) was approximately 80% in patients having low NE markers at baseline. Prior cabazitaxel or abiraterone and/or enzalutamide did not appear to affect response. A summary of the PSA and CTC responses are presented in Tables 3 and 4, respectively. Centralized assessments of images by RECIST are presented in Table 6. Additional results including statistical correlations between markers and response, median number of treatment cycles for patients in the study, and adverse events are presented in Tables 5, 7, and 8, respectively. Updated safety, tumor response and radiographic assessments from the full cohorts of 2.3 and 2.5 mg/kg are presented.

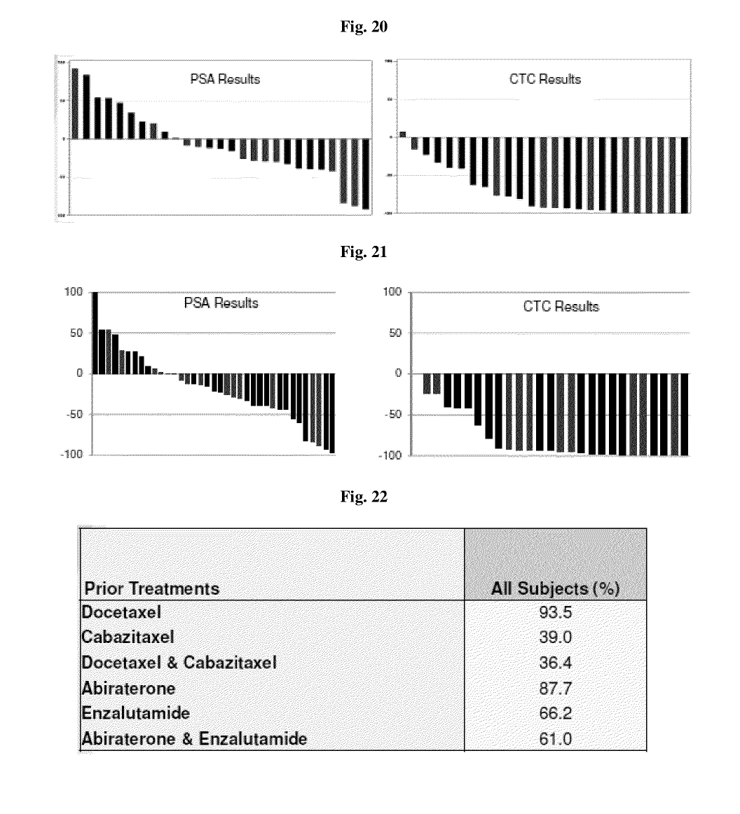

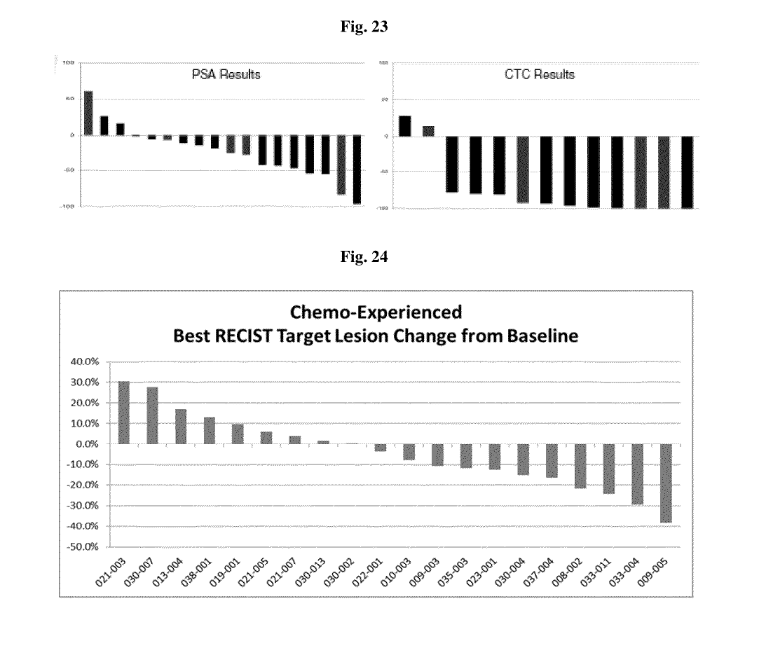

In taxane-experienced mCRPC patients treated with PSMA ADC at doses of 2.3 mg/kg, reductions of PSA≧30% were seen in approximately 36% of patients and reductions of ≧50% were seen in approximately 15% of patients (Table 3). CTC conversion from unfavorable to favorable occurred in approximately 45% of patients. PSMA expression by both IHC and CTC correlated well to PSA and CTC (p=0.019) response to PSMA ADC (Table 5; FIG. 20). Low NE markers correlated well to PSA (p=0.012) response with CTC reduction of >50% in 76% of patients (Table 5; FIG. 21). The IHC results showed that a high H-score (i.e. h-score≧200) correlated with the response to PSMA-ADC in evaluable patients (2.3 mg/kg and 2.5 mg/kg patients), as shown by declines in both PSA and CTC responses (Tables 3 and 4; FIG. 23).

PSMA ADC at 2.3 mg/kg was generally well tolerated in patients with progressive mCRPC previously treated with taxanes, and appears to be better tolerated than 2.5 mg/kg. The most common adverse events were fatigue and neutropenia. Antitumor activity, CTC and PSA reductions were observed at 2.3 and 2.5 mg/kg. Testing in taxane naïve patients is also ongoing.

TABLE 2
Summary of patient demographics and baseline characteristics

| | Dose, (n) | | |
|---|---|---|---|
| | 2.3 mg/kg | 2.5 mg/kg | |
| | (46) | (34) | All Subjects |
| Age | 70 | 71.5 | 71 |
| Race | | | |
| White | 43 (88%) | 32 (94%) | 75 (90%) |
| African American | 4 (8%) | 2 (6%) | 6 (8%) |
| Other | 2 (4%) | 0 | 2 (2%) |
| PS 0 | 17 | 13 | 30 |
| PS 1 | 28 | 20 | 48 |
| PS 2 | 4 | 1 | 5 |
| Baseline PSA | 166.5 | 312.8 | 189.3 |
| | (7.5-17459.6) | (11.2-2520.2) | (7.5-17459.6) |

TABLE 3
Summary of PSA responses

| Initial Dose Level (n) | ≧30% Decline (n) | ≧50% Decline (n) |
|---|---|---|
| 2.3 mg/kg (45) | 36.17% (17) | 14.89% (7) |
| 2.5 mg/kg (31) | 16.13% (5) | 6.45% (2) |
| PSMA Expression (26) | 34.62% (9) | 11.54% (3) |
| Low NE (39) | 35.9% (14) | 17.95% (7) |
| High IHC PSMA (18) | 38.89% (7) | 22.22% (4) |
| Total (76) | 28.21% (22) | 10.26% (9) |

TABLE 4
Summary of CTC responses

| | | Conversion ≧5 CTC at |
|---|---|---|
| | | baseline to <5 CTC |
| Initial Dose Level (n) | ≧50% Decline (n) | following treatment |
| 2.3 mg/kg (39) | 74.36% (29) | 46.88% (15/32) |
| 2.5 mg/kg (27) | 74.07% (20) | 45.45% (10/22) |
| PSMA Expression (24) | 79.17% (19) | 52.38% (11/21) |
| Low NE (29) | 75.86% (22) | 55.17% (16/29) |
| High IHC PSMA (13) | 84.62% (11) | 53.85% (7/13) |
| Total (66) | 74.24% (49) | 46.3% (25/54) |

TABLE 5
Additional results

| | | Nonparametric | |
|---|---|---|---|
| | | Correlation | |
| Marker | Response | Coefficient(n = 41) | p-value |
| NSE | Best PSA pct chg | −0.0023 | 0.9842 |
| NSE | Best CTC pct chg | 0.0620 | 0.6467 |
| CgA | Best PSA pct chg | 0.1066 | 0.3796 |
| CgA | Best CTC pct chg | 0.0497 | 0.7237 |
| % PSMA + CTC | Best PSA pct chg | 0.1126 | 0.3915 |
| % PSMA + CTC | Best CTC pct chg | −0.1022 | 0.4845 |
| PSMA Expression | Best PSA pct chg | −0.1720 | 0.2424 |
| PSMA Expression | Best CTC pct chg | −0.3517 | 0.0192* |
| Low NE | Best PSA pct chg | −0.2999 | 0.0116* |
| Low NE | Best CTC pct chg | −0.0811 | 0.5639 |
| Low NE & PSMA | Best PSA pct chg | −0.3878 | 0.0093* |
| Expression | | | |
| Low NE & PSMA | Best CTC pct chg | −0.2615 | 0.0986 |
| Expression | | | |

*p < 0.05

TABLE 6
RECIST response (RECIST 1.1)

| Total Evaluable Patients n = 15 | n | Percent |
|---|---|---|
| Progressive Disease | 8 | 16% |
| Stable Disease | 42 | 84% |

TABLE 7
Summary of treatment cycles
Median number of cycles = 4

| | Dose | Percent |
|---|---|---|
| Patients beyond 4 | 2.3 mg/kg | 38.8% |
| cycles | 2.5 mg/kg | 23.5% |

TABLE 8
Summary of adverse events grade 3 and above*

| | 2.3 mg/kg (n = 46) | | 2.5 mg/kg (n = 34) | |
|---|---|---|---|---|
| | Grades 3 and above | | Grades 3 and above | |
| Event | n | % | n | % |
| Fatigue | 9 | 18.4 | 7 | 20.6 |
| Neutropenia | 9 | 18.4 | 11 | 32.4 |
| Decreased electrolytes | 5 | 10.2 | 7 | 20.6 |
| Neuropathy peripheral | 4 | 8.2 | 2 | 5.9 |
| Anaemia | 4 | 8.2 | 3 | 8.8 |
| Dehydration | 3 | 6.1 | 1 | 2.9 |
| Asthenia | 3 | 6.1 | 2 | 5.9 |
| Muscular weakness | 2 | 4.1 | 1 | 2.9 |
| Nausea | 2 | 4.1 | 0 | 0.0 |
| Diarrhoea | 2 | 4.1 | 0 | 0.0 |
| Dyspnoea | 1 | 2.0 | 2 | 5.9 |
| Abdominal pain | 1 | 2.0 | 1 | 2.9 |
| Sepsis/Septic Shock^{±} | 0 | 0.0 | 2 | 5.9 |
| Myalgia | 0 | 0.0 | 1 | 2.9 |
| Pain | 0 | 0.0 | 1 | 2.9 |
| Abdominal pain | 0 | 0.0 | 1 | 2.9 |
| Arthralgia | 0 | 0.0 | 1 | 2.9 |

*Possibly related or greater
^{±}2 deaths occurred at 2.5 mg/kg from sepsis associated with neutropenia

TABLE 9
Discontinuations from study therapy

| | 2.3 | 2.5 |
|---|---|---|
| Reason | mg/kg (n = 46) | mg/kg (n = 34) |
| Adverse effects | 14 | 8 |
| | (28.6%) | (23.5%) |
| Disease progression | 23 | 15 |
| | (46.9%) | (44.1%) |
| Patient request | 4 | 8 |
| | (8.2%) | (23.5%) |

TABLE 10
CTC conversion rates

| | | | | Heavily pre- | |
|---|---|---|---|---|---|
| | Conversion | | | treated: post | |
| | post-treatment | | Trial | ADT & post | Clinicaltrials.gov |
| Drug | % | Study | sponsor | chemo | identifier |
| PSMA ADC | 46% | Phase 2 | Progenies | Yes | NCT01695044 |
| | (26/57) | | | | |
| Abiraterone | 41 | Phase 2 | Royal Marsden | No | NCT00474383 |
| | | | Hospital | | |
| | 34 | | MSKCC | No | NCT00485303 |
| | ~50 | Phase 3 | Cougar/J&J | No | NCT00638690 |
| Enzalutamide | 37 | Phase 1/2 | Medivation | No | NCT00510718 |
| Cabozantinib | 32 | Phase 2 | Exelixis | Yes | NCT00940225 |

### Example 4

### Biomarker Results

### Interim Analysis

It was surprisingly found that baseline levels of serum neuroendocrines, prior to treatment with PSMA ADC, correlate with efficacy in treatment using PSMA ADC (Table 5; FIG. 21). Efficacy parameters were demonstrated by a decline in baseline measures of one or more of the following: PSA levels, CTCs, PSMA⁺ CTCs and overall survival. It was observed that patients who benefited from treatment with the PSMA targeting agent, PSMA ADC, had low baseline levels of neuroendocrines and high levels of PSA (>than about 100 ng/ml) at baseline. Low neuroendocrine levels versus high neuroendocrine levels in an individual patient were calculated as follows: an individual patient's serum sample was analyzed for chromogranin A (CgA) using a radioimmunoassay and a patient value was obtained. The patient's serum sample was also analysed for neuron-specific enolase (NSE) using an enzyme immunoassay, and a patient value obtained. A patient CgA value less than three times the Upper Limit of Normal (ULN) (an average standard assay range of CgA normal values) in combination with a patient NSE value of less than one and one-half times the ULN (an average standard assay range of NSE values) is indicative of low levels of neuroendocrine biomarkers. An individual patient CgA value greater than three times the ULN in combination with the individual patient NSE value greater than one and one-half times the ULN, the individual patient is considered having high neuroendocrine biomarkers.

Patients benefiting from the PSMA ADC treatment also had greater than or equal to the median PSMA expression (median=23 MFI), in particular high density PSMA expression on PSMA⁺ CTCs at baseline, the density determined by mean fluorescence intensity (MFI) using a Cell Tracks Analyzer II® (Table 5; FIG. 20). High density was defined as >24 MFI; low density was defined as <24 MFI.

A MFI>24 (Cell Tracks Analyzer) is equivalent to a fluorescence intensity of ≧3 on a scale of zero to 4 fluorescence intensity based on visual grading using an immunofluorescence assay. A MFI<24 is equivalent to a mean fluorescence intensity of zero to <3 fluorescence intensity based on visual grading. For assays which determine the relative number of PSMA⁺ molecules/cell (see for example Wang, X et al. Mol. Cancer Ther. 10(9):2022-3 Sep. 2011, using ³H-ZJ24: GE Healthcare Life Sciences), a MFI>24 is equivalent to an average of >100,000 PSMA molecules/PSMA⁺ CTC, and a MFI<24 is equivalent to an average of <100,000 PSMA molecules/PSMA⁺ CTC.

IHC PSMA staining was scored from 0 to 3+(0, 1+, 2+, 3+) intensity with 0 being no PSMA staining and 3+ being intense PSMA staining. Each cell was assigned an intensity score with the total percentage equaling 100% (FIG. 23). A standard formula was then applied to generate an Histoscore (H-score) (Petrul et al., Molecular Cancer Therapeutics, 11(2) February 2012). The H-score is a single numerical value ranging from 0 to 300 with 0 equaling 100% of the cells described as 0 and 300 equaling 100% of the cells described as 3+. The H-score was calculated using the following formula:

H-score=(% cells showing 3+staining intensity)×3+(% cells showing 2+staining intensity)×2+(% cells showing 1+staining intensity)

The results were analyzed two different ways: a) high PSMA vs. low PSMA, and b) equal to or above the median vs. below the median.

For the first analysis, with an H-score cut off of 200, everything above or equal to 200 was considered “high IHC PSMA,” and everything below 200 was considered “low IHC PSMA.”

### Example 5

### Summary of Phase 2 Clinical Trial

### Conclusion of Clinical Results and Biomarker Results

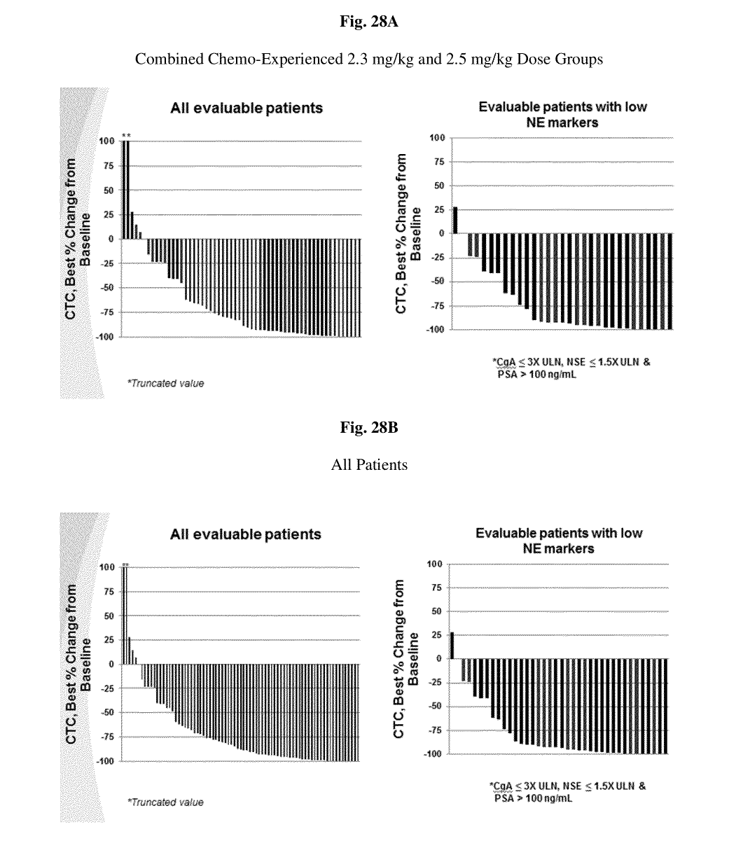

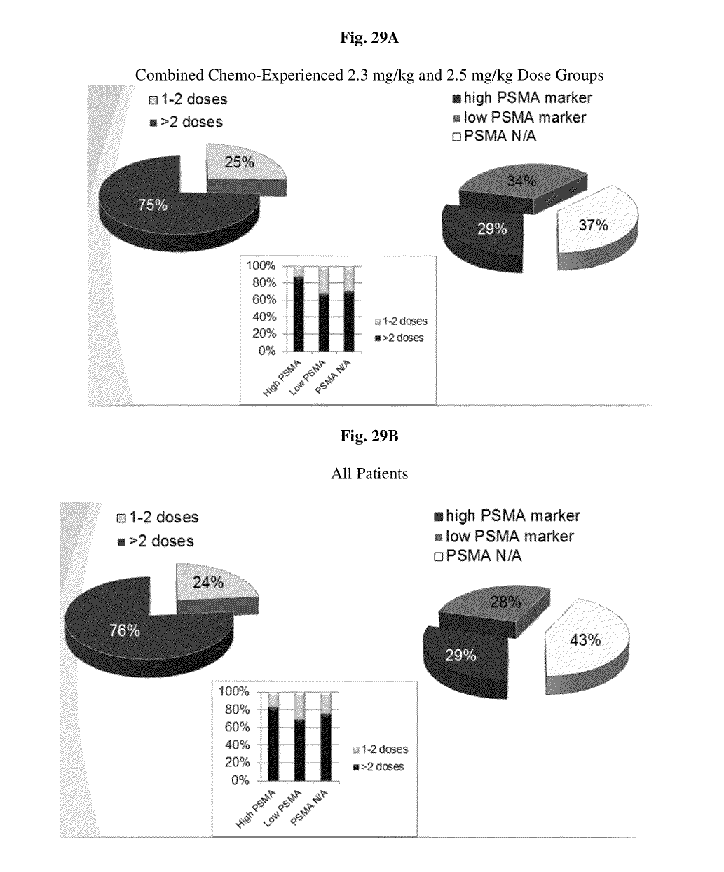

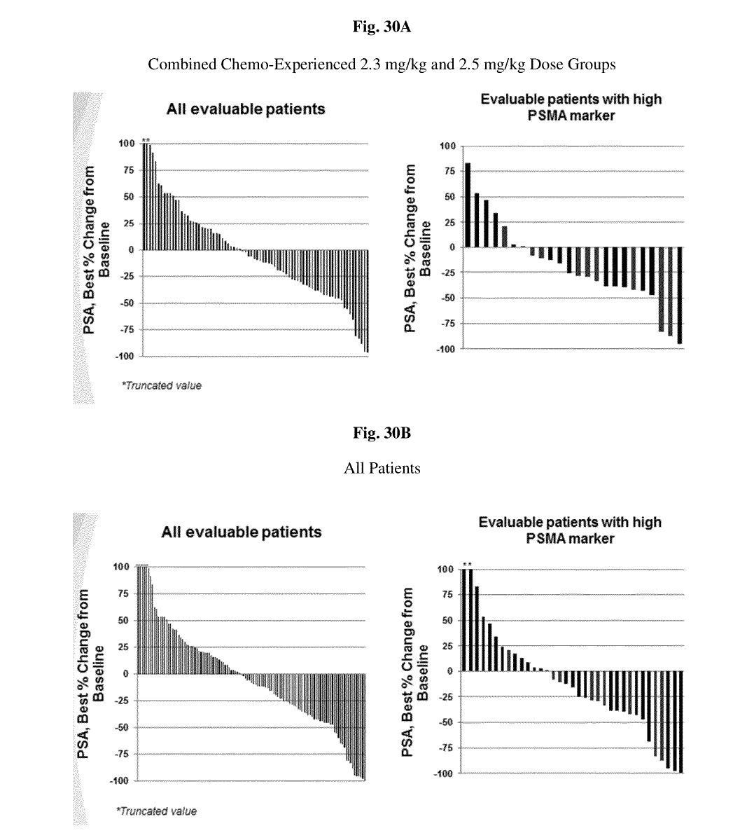

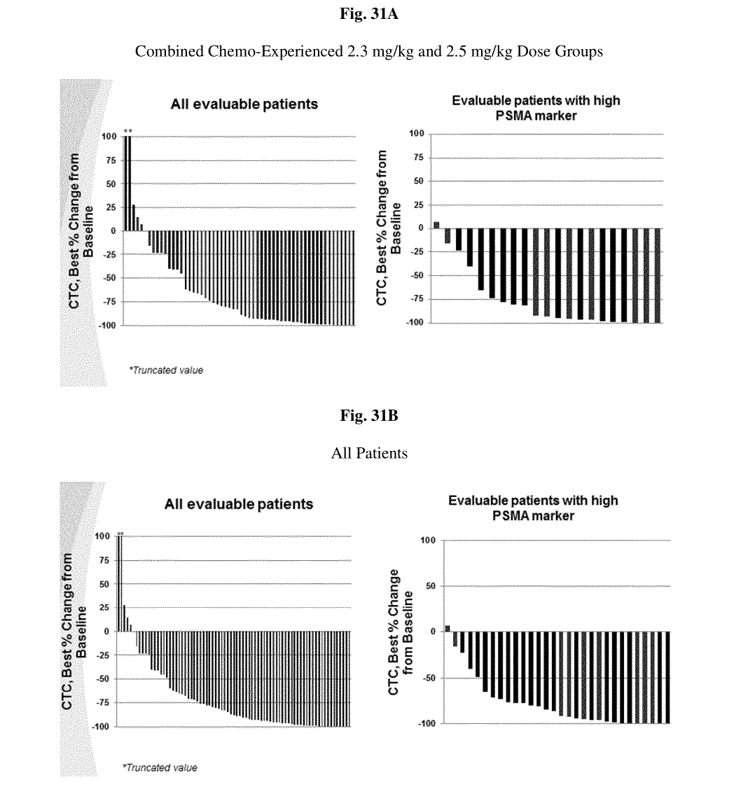

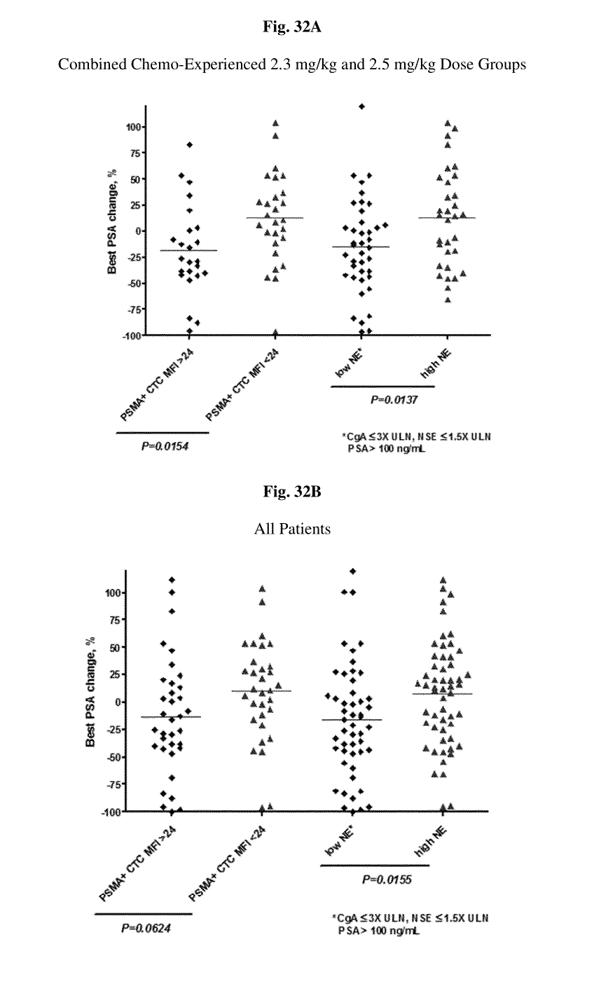

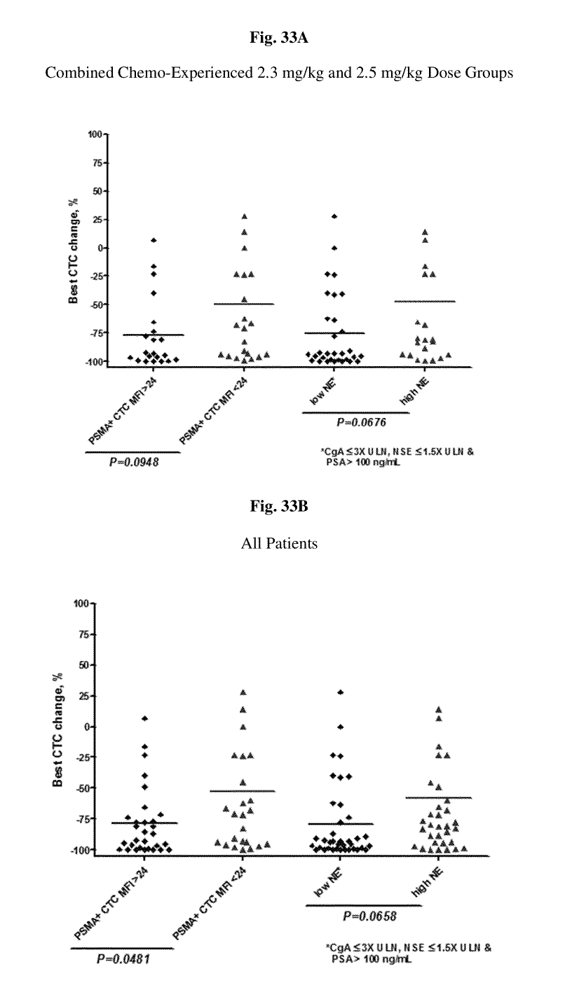

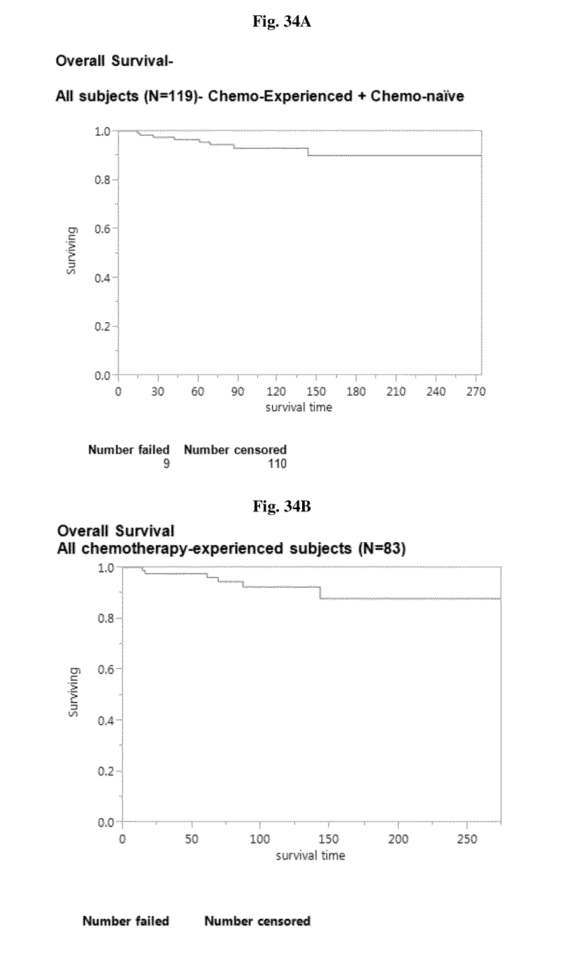

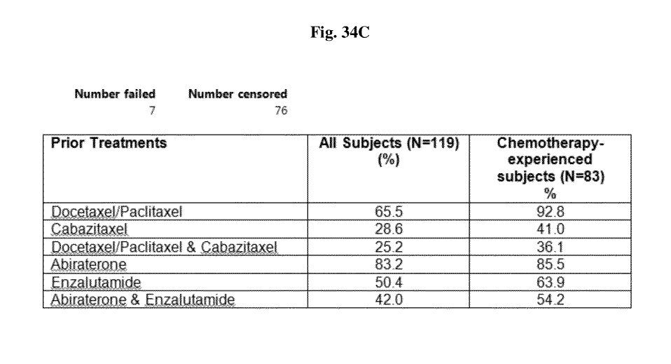

The studies according to Examples 3 and 4 as provided above were continued. Data representing the final (end of study) results are provided in the below tables. Treatment with PSMA ADC provided significant anti-tumor activity in mCRPC patients who had previously progressed despite taxane-containing chemotherapy and treatment with abiraterone and/or enzalutamide. CTC responses of >50% in 78% of the patients (94% after biomarker application); CTC conversion in 47% of patients (75% after biomarker application); and PSA reduction>30% in 30% of patients (53% after biomarker application). Biomarkers identified and confirmed by this trial provide a clear strategy for the tailoring of therapy to patients. FIGS. 26A-34C provide graphs of data obtained upon completion of the Phase 2 clinical trial. The algorithm for determining low NE v. high NE for end of Phase 2 trial data is as follows: CgA patient assay value≦3×ULN; and in combination NSE patient assay value≦1.5×ULN; equals low neuroendocrine levels.

TABLE 11
Summary of patient demographics and baseline characteristics

| | Dose, (n) | | |
|---|---|---|---|
| | 2.3 mg/kg | 2.5 mg/kg | |
| | [symbol] | (34) | All Subjects |
| Median Age | 70 | 71.5 | 71 |
| Race | | | |
| White | 43 (88%) | 32 (94%) | 75 (90%) |
| African American | 4 (8%) | 2 (6%) | 6 (7%) |
| Other | 2 (4%) | 0 | 2 (2%) |
| PS 0 | 17 | 13 | 30 |
| PS 1 | 28 | 20 | 48 |
| PS 2 | 4 | 1 | 5 |
| Median Baseline | 166.5 | 312.8 | 189.3 |
| PSA (Range) | (7.5-17459.6) | (11.2-2520.2) | (7.5-17459.6) |

[symbol]

indicates data missing or illegible when filed

TABLE 12
Summary of PSA responses

| Initial Dose Level (n) | ≧30% Decline (n) | ≧50% Decline (n) |
|---|---|---|
| 2.3 mg/kg (47) | 38.30% (18) | 14.89% (7) |
| 2.5 mg/kg (31) | 25.80% (5) | 6.45% (2) |
| High CTC PSMA (24) | 41.67% (10) | 12.50% (3) |
| Low NE (40) | 35.00% (14) | 17.50% (7) |
| High IHC PSMA (18) | 38.89% (7) | 22.22% (4) |
| Total (78) | 29.49% (23) | 11.54% (9) |

TABLE 13
Summary of CTC responses

| | | Conversion ≧5 CTC |
|---|---|---|
| | | at baseline to <5 CTC |
| Initial Dose Level (n) | ≧50% Decline (n) | following treatment |
| 2.3 mg/kg (35) | 77.14% (27) | 42.86% (15/35) |
| 2.5 mg/kg (23) | 69.57% (16) | 47.83% (11/23) |
| High CTC PSMA (21) | 80.95% (17) | 52.38% (11/21) |
| Low NE (31) | 77.42% (24) | 51.61% (16/31) |
| High IHC PSMA (15) | 86.67% (13) | 53.33% (8/15) |
| Total (58) | 74.14% (43) | 44.83% (26/58 |

TABLE 14
Additional results

| | | Nonparametric | |
|---|---|---|---|
| | | Correlation | |
| Marker | Response | Coefficient(n = 55) | p-value |
| NSE | Best PSA pct chg | −0.0376 | 0.7435 |
| NSE | Best CTC pct chg | 0.0596 | 0.6508 |
| CgA | Best PSA pct chg | 0.0895 | 0.4547 |
| CgA | Best CTC pct chg | −0.0371 | 0.7880 |
| High CTC PSMA | Best PSA pct chg | −0.2159 | 0.0576 |
| High CTC PSMA | Best CTC pct chg | −0.1460 | 0.2656 |
| High IHC PSMA | Best PSA pct chg | −0.2514 | 0.0264 * |
| High IHC PSMA | Best CTC pct chg | −0.1859 | 0.1549 |
| Low NE | Best PSA pct chg | −0.2324 | 0.0406 * |
| Low NE | Best CTC pct chg | −0.2235 | 0.0860 |
| Low NE & High | Best PSA pct chg | −0.3041 | 0.0068 * |
| CTC PSMA | | | |
| Low NE & High | Best CTC pct chg | −0.2903 | 0.0245 * |
| CTC PSMA | | | |

* p < 0.05

TABLE 15
RECIST response (RECIST 1.1)

| Total Evaluable Patients (n = 60) | n | Percent |
|---|---|---|
| Progressive Disease | 11 | 18% |
| Stable Disease | 49 | 82% |

TABLE 16
Summary of treatment cycles
Median number of cycles = 4

| | Dose | Percent |
|---|---|---|
| Patients beyond 4 | 2.3 mg/kg | 40.8% |
| cycles | 2.5 mg/kg | 23.5% |

TABLE 17
Summary of adverse events grade 3 and above*

| | 2.3 mg/kg (n = 49) | | 2.5 mg/kg (N = 34) | |
|---|---|---|---|---|
| Event | n | % | n | % |
| Neutropenia | 9 | 18.37 | 12 | 35.29 |
| Fatigue | 10 | 20.41 | 7 | 20.59 |
| Decreased electrolytes | 6 | 12.24 | 7 | 20.59 |
| Anaemia | 5 | 10.20 | 3 | 8.82 |
| Neuropathy peripheral | 4 | 8.16 | 2 | 5.88 |
| Asthenia | 3 | 6.12 | 2 | 5.88 |
| Dehydration | 3 | 6.12 | 1 | 2.94 |
| Muscular weakness | 3 | 6.12 | 1 | 2.94 |
| White blood cell count | 2 | 4.08 | 2 | 5.88 |
| decreased | | | | |
| Dyspnoea | 1 | 2.04 | 2 | 5.88 |
| Abdominal pain | 1 | 2.04 | 1 | 2.94 |
| Diarrhoea | 2 | 4.08 | 0 | 0.00 |
| Hyperglycaemia | 1 | 2.04 | 1 | 2.94 |
| Ileus | 2 | 4.08 | 0 | 0.00 |
| Leukopenia | 0 | 0.00 | 2 | 5.88 |
| Lipase increased | 1 | 2.04 | 1 | 2.94 |
| Metabolic acidosis | 0 | 0.00 | 2 | 5.88 |
| Nausea | 2 | 4.08 | 0 | 0.00 |
| Sepsis/Septic shock^{±} | 0 | 0.00 | 2 | 5.88 |
| Supraventricular tachycardia | 0 | 0.00 | 2 | 5.88 |
| Arthralgia | 0 | 0.00 | 1 | 2.94 |
| Atrial fibrillation | 0 | 0.00 | 1 | 2.94 |
| Bacteraemia | 0 | 0.00 | 1 | 2.94 |
| Blood creatine phosphokinase | 0 | 0.00 | 1 | 2.94 |
| increased | | | | |
| Blood lactic acid increased | 0 | 0.00 | 1 | 2.94 |
| Bone pain | 1 | 2.04 | 0 | 0.00 |
| Chronic obstructive | 1 | 2.04 | 0 | 0.00 |
| pulmonary disease | | | | |
| Diabetic ketoacidosis | 1 | 2.04 | 0 | 0.00 |
| Dysphagia | 1 | 2.04 | 0 | 0.00 |
| Electrocardiogram QT | 0 | 0.00 | 1 | 2.94 |
| prolonged | | | | |
| Gait disturbance | 1 | 2.04 | 0 | 0.00 |
| International normalised ratio | 0 | 0.00 | 1 | 2.94 |
| increased | | | | |
| Iron deficiency anaemia | 1 | 2.04 | 0 | 0.00 |
| Liver function test abnormal | 0 | 0.00 | 1 | 2.94 |
| Lobar pneumonia | 0 | 0.00 | 1 | 2.94 |
| Lymphocyte count decreased | 0 | 0.00 | 1 | 2.94 |
| Myalgia | 0 | 0.00 | 1 | 2.94 |
| Myocardial infarction | 0 | 0.00 | 1 | 2.94 |
| Non-cardiac chest pain | 0 | 0.00 | 1 | 2.94 |
| Pain | 1 | 2.04 | 0 | 0.00 |
| Pancreatitis | 0 | 0.00 | 1 | 2.94 |
| Pleural effusion | 1 | 2.04 | 0 | 0.00 |
| Pneumonia primary atypical | 1 | 2.04 | 0 | 0.00 |
| Rash | 0 | 0.00 | 1 | 2.94 |
| Sinus tachycardia | 1 | 2.04 | 0 | 0.00 |
| Thrombocytopenia | 1 | 2.04 | 0 | 0.00 |
| Urinary retention | 0 | 0.00 | 1 | 2.94 |
| Urinary tract infection | 1 | 2.04 | 0 | 0.00 |
| Vomiting | 1 | 2.04 | 0 | 0.00 |

*Possibly related or greater
^{±}2 deaths occurred at 2.5 mg/kg from sepsis associated with neutropenia

TABLE 18
Discontinuations from study therapy

| | 2.3 | 2.5 |
|---|---|---|
| Reason | mg/kg (n = 49) | mg/kg (n = 34) |
| Adverse effects | 16 | 8 |
| | (32.7%) | (23.5%) |
| Disease progression | 23 | 15 |
| | (46.9%) | (44.1%) |
| Patient request | 3 | 8 |
| | (6.1%) | (23.5%) |

### REFERENCES

1. Siegel R, Naishadham D, Jemal A. Cancer statistics, 2012. CA Cancer J Clin 2012; 62:10-29.
2. Stamey T A, Yemoto C M, McNeil J E, Sigal B M, et al. Prostate cancer is highly predictable: a prognostic equation based on all morphological variables in radical prostatectomy specimens. J Urol 2000; 163:1155-1160.
3. El-Amm J, Aragon-Ching J B. The changing landscape in the treatment of metastatic castration-resistant prostate cancer. Ther Adv Med Oncol. 2013 January; 5(1):25-40. doi: 10.1177/1758834012458137. PubMed PMID: 23323145; PubMed Central PMCID: PMC3539272.
4. Beltran H, Rickman D S, Park K, Chae S S, Sboner A, MacDonald T Y, et al. Molecular characterization of neuroendocrine prostate cancer and identification of new drug targets. Cancer Discov 2011; 1:487-95.
5. Hirano D, Okada Y, Minei S, Takimoto Y, Nemoto N. Neuroendocrine differentiation in hormone refractory prostate cancer following androgen deprivation therapy. Eur Urol 2004; 45:586-92.
6. Kamiya N, Suzuki H, Kawamura K, Imamoto T, Naya Y, Tochigi N, et al. Neuroendocrine differentiation in stage D2 prostate cancers. Int J Urol 2008; 15:423-8.
7. Komiya A, Suzuki H, Imamoto T, Kamiya N, Nihei N, Naya Y, et al. Neuroendocrine differentiation in the progression of prostate cancer. Int J Urol 2009; 16:37-44.
8. Matei D V, Renne G, Pimentel M, Sandri M T, Zorzino L, Botteri E, et al. Neuroendocrine differentiation in castration-resistant prostate cancer: a systematic diagnostic attempt. Clin Genitourin Cancer 2012; 10:164-73.
9. Appetecchia M, Mecule A, Pasimeni G, Iannucci C, DeCarli P et al. Incidence of high chromogranin A serum levels in patients with non metastatic prostate adenocarcinoma. J Exp. & Clin. Cancer Res. 2010, 29:166
10. Beltran H, Tagawa S T, Park K, MacDonald T, Milowsky M I, Mosquera J M, et al. Challenges in recognizing treatment-related neuroendocrine prostate cancer. J Clin Oncol 2012; 30:e386-e389.

Each of the foregoing patents, patent applications and references that are recited in this application are herein incorporated in their entirety by reference. However, such recitation is not intended to be an admission that any of the foregoing patents, patent applications and references is a prior art reference. Having described the presently preferred embodiments, and in accordance with the present invention, it is believed that other modifications, variations and changes will be suggested to those skilled in the art in view of the teachings set forth herein. It is, therefore, to be understood that all such variations, modifications, and changes are believed to fall within the scope of the present invention as defined by the appended claims.

I/we claim:

1. A companion diagnostic test comprising:
a. obtaining one or more biological samples from a subject undergoing a treatment or considered for a treatment;
b. assaying a panel of biomarkers;
c. generating a score with an algorithm based on the assay results of said panel of biomarkers; and
d. determining the likely responsiveness of said subject to said treatment based on the score.

2. The companion diagnostic test of claim 1, wherein at least one of the biological samples is obtained at baseline or prior to treatment.

3. (canceled)

4. The companion diagnostic test of claim 1, wherein the panel of biomarkers comprises serum neuroendocrine markers.

5. (canceled)

6. The companion diagnostic test of claim 1, wherein the algorithm comprises:
(a) CgA subject assay value≦3×(Upper Limit of Normal (ULN) and
NSE subject assay value≦1.5 ULN, equals low neuroendocrine levels;
(b) CgA subject assay value≦3×5 nmole/L and
NSE subject assay value≦1.5×12.5 ng/mL, equals low neuroendocrine levels;
(c) CgA subject assay value>3×ULN and
NSE subject assay value>1.5 ULN, equals high neuroendocrine levels; or
(d) CgA subject assay value>3×5 nmole/L and
NSE subject assay value>1.5×12.5 ng/mL, equals high neuroendocrine levels.

7-9. (canceled)

10. The companion diagnostic test of claim 1, wherein the panel of biomarkers further comprises a Prostate Serum Antigen (PSA) assay.

11. The companion diagnostic test of claim 10, wherein the PSA algorithm comprises:
PSA value>100 ng/mL, equals high PSA; or
PSA value<100 ng/mL, equals low PSA.

12. (canceled)

13. The companion diagnostic test of claim 1, wherein the algorithm comprises:
CgA subject assay value≦3×ULN and
NSE subject assay value≦1.5 ULN, equals low neuroendocrine levels; and
PSA value>100 ng/mL.

14. The companion diagnostic test of claim 1, wherein the panel of biomarkers further comprises prostate-specific membrane antigen (PSMA) intensity, wherein the intensity of PSMA is determined with an immunohistochemical (IHC) procedure and determining an H-score.

15. The companion diagnostic test of claim 14, wherein the H-score is calculated according to the following formula:
H-score=(% cells showing 3+staining intensity)×3+(% cells showing 2+staining intensity)×2+(% cells showing 1+staining intensity).

16. The companion diagnostic test of claim 15, where the algorithm comprises:
H-score is ≧200.

17. The companion diagnostic test of claim 16, wherein an H-score ≧200 equals high PSMA intensity.

18. (canceled)

19. The companion diagnostic test of claim 1, wherein the algorithm comprises:
CgA subject assay value≦3×ULN and
NSE subject assay value≦1.5 ULN, equals low neuroendocrine levels;
PSA value>100 ng/mL; and
H-score is ≧200.

20. The companion diagnostic test of claim 1, wherein the panel of biomarkers further comprises Circulating Tumor Cells (CTCs) assay.

21-23. (canceled)

24. The companion diagnostic test of claim 1, wherein the panel of biomarkers further comprises an assay of cell surface PSMA density.

25. The companion diagnostic test of claim 24, wherein the algorithm comprises:
cell surface PSMA density>100,000 molecules of PSMA/PSMA⁺ CTC, equals high cell surface PSMA density; or
cell surface PSMA density>3+ average cell fluorescence intensity on a scale of zero to 4+ fluorescence intensity, equals high cell surface PSMA density, and the neuroendocrine level is low.

26. (canceled)

27. The companion diagnostic test of claim 24 any one of claims 24-26, wherein the cell surface PSMA density is measured by mean fluorescence intensity (MFI).

28. (canceled)

29. The companion diagnostic test of claim 27, wherein the algorithm comprises:
MFI>24, equals high cell surface PSMA density, and the neuroendocrine level is low.

30. The companion diagnostic test of claim 1, wherein a score at baseline of
(a) low neuroendocrine levels, and
high PSA,
is indicative of likely responsiveness to treatment; or
(b) low neuroendocrine levels,
high PSA, and
high PSMA intensity or high cell surface PSMA density on PSMA⁺ CTC or tumor tissue,
is indicative of likely responsiveness to treatment.

31-60. (canceled)

61. A method of treating metastatic prostate cancer comprising:
a) performing a biomarker test on a patient at baseline; and
b) providing a treatment to the patient according to the results of the biomarker test.

62-96. (canceled)

97. A biomarker assay for identifying a subject likely to respond to a PSMA targeted therapy comprising: determining the average cell surface PSMA density on PSMA⁺ CTCs.

98-130. (canceled)

131. A biomarker assay for identifying a subject likely to respond to a PSMA targeted therapy comprising: measuring one or more serum neuroendocrine markers in a sample from a subject.

132-142. (canceled)

143. A diagnostic kit for selecting a prostate cancer patient for treatment by a PSMA ligand-anticancer agent conjugate, wherein the diagnostic kit comprises;
assay reagents to measure serum levels of neuroendocrine enzymes and instructions for selecting;
reagents for use in an biomarker assay to determine the PSMA density on PSMA⁺ CTCs in a biological sample obtained from the patient, and instructions for selecting; or
reagents for use in an IHC assay to determine the H-score of a biological sample obtained from the patient, and instructions for selecting.

144-161. (canceled)
